(19) United States
(12) Patent Application Publication
(10) Pub. No.: US 2024/0368557 A1
(43) Pub. Date: Nov. 7, 2024

(54) STEM CELL-DERIVED ALPHA CELLS AND METHODS OF GENERATING SAME

(71) Applicant: President and Fellows of Harvard College, Cambridge, MA (US)

(72) Inventors: Douglas A. Melton, Cambridge, MA (US); Quinn P. Peterson, Cambridge, MA (US); Adrian Veres, Cambridge, MA (US)

(21) Appl. No.: 18/773,343

(22) Filed: Jul. 15, 2024

Related U.S. Application Data
(62) Division of application No. 17/054,114, filed on Nov. 9, 2020.
(60) Provisional application No. 62/692,778, filed on Jun. 30, 2018.
(60) Provisional application No. 62/668,208, filed on May 7, 2018.

(51) Int. Cl. C12N 5/071

(52) U.S. Cl. CPC C12N 5/0678; C12N 5/0676; C12N 2501/15; C12N 2501/727; C12N 2501/999; C12N 2506/02; C12N 2506/45

(57) ABSTRACT

Disclosed herein are methods, differentiation protocols and compositions useful for inducing a cell maturation, and isolated populations of SC-α cells for use in various applications, such as cell therapy.

### CROSS-REFERENCE TO RELATED APPLICATIONS

This application is divisional of U.S. application Ser. No. 17/054,114, filed Nov. 9, 2020, which is a national stage filing under 35 U.S.C. 371 of International Application No. PCT/US2019/031214, filed May 7, 2019, which claims the benefit of U.S. Provisional Application No. 62/668,208, filed on May 7, 2018, and U.S. Provisional Application No. 62/692,778, filed on Jun. 30, 2018. The entire teachings of the above applications are incorporated herein by reference. International Application No. PCT/US2019/031214 was published under PCT Article 21(2) in English.

### BACKGROUND OF THE INVENTION

Diabetes mellitus is characterized by dysfunction of the pancreatic endocrine cells either due to autoimmune attack or loss of proper functional responses. Although diabetes primarily involves the beta cell, there is mounting evidence that alpha cell dysfunction plays a key role in disease etiology^{(1,2)}. Furthermore, a significant complication of type 1 diabetes is hypoglycemia. Patients with type 1 diabetes must cope with significant fluctuation in blood glucose levels including acute hypoglycemia⁽³⁾. Without immediate intervention, acute hypoglycemia can lead to decreased neurological function, seizure, coma and eventually death⁽⁴⁾.

In the normal pancreas, hormone expressing endocrine cells function within the islets of Langerhans to precisely regulate blood glucose and energy metabolism. Under hypoglycemic conditions, alpha cells in the islet protect against hypoglycemia by secreting the hormone glucagon in response to low blood sugar⁽⁵⁾. Glucagon raises blood glucose by increasing glycolysis and gluconeogenesis in the liver^{(6,7)}. Although alpha cells persist in diabetic islets, these islets are not capable of mounting an appropriate glucagon response, perhaps due to alpha cell-beta cell interactions that are absent in the islets of type 1 diabetics⁽⁸⁾. Recent studies have implicated dysfunction in alpha cells as contributing factors in the elevated blood glucose levels observed in diabetic patients^{(5, 8)}.

Despite mounting evidence regarding the importance of alpha cells in proper function of the islet, little work toward the development of protocols for generating these cells has been reported. Rezania, et al. reported in 2011 a protocol for generating alpha cells through a polyhormonal intermediate⁽⁹⁾. Additionally, several efforts have reported the conversion of various cell types into alpha cells via transdifferentiation. Despite these early reports, these protocols have not been reproduced or been widely adopted by the field. Nevertheless, new protocols and methods for generating stem cell derived alpha cells are needed.

### SUMMARY OF THE INVENTION

A novel, efficient and scalable method for the generation of stem cell-derived alpha (SC-alpha) cells is provided herein. Starting with a polyhormonal intermediate, an optimized protocol for the generation of these stem cell derived polyhormonal cells is established. These cells are shown to closely resemble alpha cells rather than beta cells. While these polyhormonal cells express pro-insulin, they do not proteolytically process nor secrete mature insulin. Further, using a phenotypic screen setup, a small molecule is identified that is capable of driving these polyhormonal cells to an alpha cell identity and reduce their proinsulin expression. The resulting directed differentiation protocol produces SC-alpha cells that express markers of mature alpha cells, respond to glucagon secretagogues and elicit a physiological response when transplanted in mice.

In some aspects the disclosure provides a stem cell-derived alpha cell.

In some embodiments the cell expresses one or more of the following genes: ARX, IRX1, IRX2, DPP4, GCG, PSCK2, Pou6F2, FEV, TTR, and GC. In some embodiments the cell co-expresses ARX, IRX1, and IRX2. In some embodiments the expression of the genes is enriched relative to in vivo pancreatic populations. In some embodiments the cell secretes glucagon in response to glucose and/or glucagon secretagogues. In some embodiments the cell does not express c-peptide. In some embodiments the cell is monohormonal. In some embodiments the cell comprises one or more glucagon granules. In some embodiments the cell exhibits an ultrastructure similar to cadaveric alpha cells.

In some embodiments the cell is differentiated in vitro from an endocrine cell, a pancreatic progenitor cell, or a pluripotent stem cell. In some embodiments the pancreatic progenitor cell is selected from the group consisting of a Nkx6-1-positive pancreatic progenitor cell, a Pdx1-positive pancreatic progenitor cell, and a Nkx6-1-positive, Pdx1-positive pancreatic progenitor cell. In some embodiments the pluripotent stem cell is selected from the group consisting of an embryonic stem cell and induced pluripotent stem cell. In some embodiments the cell is human.

In some aspects the disclosure provides a cell line comprising one or more SC-α cells as described herein. In some aspects the disclosure provides an SC-islet comprising one or more SC-α cells as described herein. In some embodiments the SC-islet further comprises one or more of SC-β cells and SC-δ cells.

In some aspects the disclosure provides methods of producing SC-alpha cells from polyhormonal cells. The method comprises contacting a population of cells comprising a polyhormonal cell with a PKC activator to induce the maturation of at least one polyhormonal cell in the population into at least one SC-alpha cell.

In some embodiments the PKC activator comprises PdbU. In some embodiments the SC-alpha cells express and secrete glucagon in response to glucose and/or glucagon secretegogues. In some embodiments the SC-alpha cells exhibit an ultrastructure similar to cadaveric alpha cells.

In some aspects the disclosure provides methods of producing SC-alpha cells from stem cells, in vitro. The method comprises contacting a population of cells comprising a gut tube cell with i) at least one bone morphogenic protein (BMP) signaling pathway inhibitor and ii) at least one retinoic acid (RA) signaling pathway activator, to induce the differentiation of at least some of the gut tube cells into pancreatic progenitor cells; contacting a population of cells comprising a pancreatic progenitor cell with at least one BMP signaling pathway inhibitor, to induce the differentiation of at least some of the pancreatic progenitor cells into endocrine progenitor cells; contacting a population of cells comprising a endocrine progenitor cell with at least one TGF-β signaling pathway inhibitor, to induce the differentiation of at least some of the endocrine progenitor cells into polyhormonal cells; and contacting a population of cells comprising a polyhormonal cell with a PKC activator to induce the maturation of at least one polyhormonal cell into at least one SC-alpha cell.

In some embodiments, the BMP signaling pathway inhibitor comprises LDN193189. In some embodiments, the RA signaling pathway activator comprises retinoic acid. In some embodiments the TGF-β signaling pathway inhibitor comprises Alk5 inhibitor II. In some embodiments the PKC activator comprises PdbU.

In some aspects the disclosure provides methods for directing differentiation of a population of cells. The methods comprise inhibiting expression of a regulator of cell fate during a differentiation protocol, wherein the regulator is PAX4, thereby directing differentiation of a population of cells towards SC-α cells.

In some aspects the disclosure provides methods for directing differentiation of a population of cells. The methods comprise inhibiting expression of a first regulator of cell fate during a differentiation protocol, wherein the first regulator is PAX4, and activating expression of a second regulator of cell fate during a differentiation protocol, wherein the second regulator is ARX, thereby directing differentiation of a population of cells towards SC-α cells.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1G demonstrate polyhormonal cells in endocrine differentiations. FIG. 1A shows differentiation to SC-beta cells results in a heterogeneous population. In this differentiation, 9.4% of cells co-express markers for insulin and glucagon expression GLP2 and c-peptide. FIG. 1B provides a schematic of a directed differentiation protocol from hPSC into polyhormonal cells, which results in >60% glucagon expressing cells (FIG. 1C). FIG. 1D shows immunofluorecent staining of a cluster for pro/insulin and pro/glucagon. FIG. 1E shows single cell RNAseq analysis of polyhormonal cells obtained from the SC-beta protocol. The expression profile of these polyhormonal cells is similar to the expression profile of human islet alpha cells (FIG. 1F). Polyhormonal cells secrete glucagon, but do not secrete insulin (FIG. 1G). The left panel provides representative ELISA measurements of secreted glucagon from HUES8 differentiated cells using the polyhormonal protocol or human islets challenged sequentially with 2.8 or 20 mM glucose with a 30-minute incubation for each concentration. The right panel provides representative ELISA measurements of secreted full processed human insulin from HUES8 differentiated polyhormonal cells and human islets challenged sequentially with 2.8 or 20 mM glucose with a 30-minute incubation for each concentration.

FIGS. 2A-2B show pro/insulin expression is reduced upon transplantation and extended culture in vitro. FIG. 2A shows expression of pro/insulin and pro/glucagon in grafts after transplantation of polyhormonal cells under the kidney capsule of mice at 14 days (left), 28 days (middle) and 56 days (right) post-transplant. Each inlay shows a zoomed in view of cells at each time point. Co-expression of pro/insulin and pro/glucagon is observed at 14 days. At 28 days pro/insulin expression is absent from the majority of cells. FIG. 2B shows extended culture in vitro results in a fraction of polyhormonal cells reducing pro/insulin expression and continuing to express pro/glucagon. After 28 days extended culture, 23% of cells express pro/glucagon but not pro/insulin.

FIGS. 3A-3F demonstrate use of a screen to identify compounds that promote alpha cell identity by evaluating expression of pro/insulin and pro/glucagon. Small molecules targeting known pathways (43 compounds) were incubated with polyhormonal cells in quintuplicate for 96 hours. FIGS. 3A-3B provide primary screening results showing the percentage of cells expressing both pro/insulin and pro/glucagon (FIG. 3A) and the total number of cells expressing pro/glucagon (FIG. 3B). The PKC activator PdbU significantly reduced the percentage of polyhormonal cells and increased the percentage of cells expressing pro/glucagon. FIG. 3C shows flow cytometry results showing the effect of the PKC activator PdbU on polyhormonal cells at stage 6 day 28. Immnofluorecence of clusters treated with and without PdbU for 28 days (FIG. 3D). FIG. 3E shows additional PKC activators were evaluated for their effect on polyhormonal cells. All PKC activators increased the percentage of SC-alpha cells. FIG. 3F shows that Stage 6 of the protocol converts polyhormonal cells into SC-alpha cells.

FIGS. 4A-4F provide characterization of SC-alpha cells. FIGS. 4A-4B show that the ultra-structure and granule morphology of SC-alpha cells (FIG. 4A) is similar to the morphology of human cadaveric alpha cells (FIG. 4B). FIG. 4C demonstrates SC-alpha cells secrete glucagon in response to glucose, are inhibited by somatostatin, and increase glucagon secretion upon treatment with known secretagogues, ranolazine and veratridine. Upon transplantation, SC-alpha cells prevent hypoglycemia in mice. FIG. 4D shows that continuous glucose monitoring of mice for 4 weeks reveals that mice transplanted with SC-alpha cells (n=9) have elevated blood glucose compared to control animals (n=9). The graph represents the average daily blood glucose value at 5-minute intervals for all animals in each treatment group. Each data point represents the average of 252 blood glucose readings. FIG. 4E shows percentage of time over 4 weeks spent in range (≥70 mg/dl) and hypoglycemic (<70 mg/dl). FIG. 4F shows protection from acute hypoglycemia. Representative glucose reading for 2 mice injected with insulin.

FIG. 5 shows continuous glucose monitoring of mice transplanted with SC-alpha cells or controls challenged with an injection of exogenous insulin at 15 hours. Top left panel excerpted from FIG. 4 and remaining panels provide replicates with additional mice.

FIG. 6 provides flow cytometry of the iPS cell line 1016 differentiated with the SC-alpha cell protocol. Modifications to the concentration of LDN and PdbU were necessary to adapt to this iPS cell line.

FIG. 7 provides flow cytometry of HUES8 cells differentiated to SC-beta cells and treated with PdbU for 14 days during stage 6. PdbU treatment did not effect the percentage of Nkx6.1/C-peptide co-positive cells.

FIG. 8 provides an analysis of continuous glucose monitoring data from mice transplanted with SC-alpha cells or control animals during normal feeding and activity. Circadian rhythmicity in blood glucose concentration is maintained upon transplantation of SC-alpha cells as shown by raw (top) and smoothend (middle) averages of blood glucose for each cohort. The periodicity (bottom) is not significantly modified upon transplantation of SC-alpha cells.

FIGS. 9A-9C demonstrate results of using PdbU during differentiation. FIG. 9A provides a heatmap of key alpha cell marker transcripts and expression levels in SC-alpha cells compared to endocrine cell types from human islets. FIG. 9B provides a tSNE plot of cells differentiated with PdbU and without PdbU demonstrated no significant difference in gene signature of cells generated with the addition of PdbU despite specific differences in processing and expression of pro/insulin and pro/glucagon. FIG. 9C shows differential expression analysis of SC-alpha cells derived in the presence and absence of PdbU. Differentially expressed genes are marked in red and are listed in FIG. 12.

FIGS. 10A-10B demonstrate transplantation of SC-alpha cells results in elevated fasting blood glucose levels and elevated fasting glucagon levels in mice. FIG. 10A shows average fasting blood glucose in mice transplanted with SC-alpha cells or control animals. FIG. 10B shows average fasting glucagon levels in mice transplanted with SC-alpha cells or control animals.

FIG. 11 shows demonstrates withdrawal of PdbU. SC-alpha cells derived with PdbU and control conditions were cultured in the absence of PdbU for 7 days to determine the stability of the monohormonal phenotype. Withdrawal of PdbU for 7 days did not significantly reduce the percentage of monohormonal SC-alpha cells.

FIG. 12 provides a list of differentially expressed transcripts between SC-alpha cells derived with and without PdbU treatment.

### DETAILED DESCRIPTION OF THE INVENTION

Aspects of the disclosure relate to methods for producing stem cell-derived alpha cells (SC-α) cells. Other aspects of the disclosure relate to stem cell-derived alpha (SC-α) cells and uses thereof. Still other aspects of the disclosure relate to methods for identifying, distinguishing and enriching for sub-populations of cells contained within populations of polyhormonal cells, as well as methods of directing the differentiation of cells with multiple potential differentiation outcomes toward or away from particular differentiation outcomes.

For convenience, certain terms employed herein, in the specification, examples and appended claims are collected here. Unless otherwise defined, all technical and scientific terms used herein have the same meaning as commonly understood by one of ordinary skill in the art to which this invention belongs.

The term “differentiated cell” is meant any primary cell that is not, in its native form, pluripotent as that term is defined herein. Stated another way, the term “differentiated cell” refers to a cell of a more specialized cell type derived from a cell of a less specialized cell type (e.g., a stem cell such as an induced pluripotent stem cell) in a cellular differentiation process. Without wishing to be limited to theory, a pluripotent stem cell in the course of normal ontogeny can differentiate first to an endoderm cell that is capable of forming pancreas cells and other endoderm cell types. Further differentiation of an endoderm cell leads to the pancreatic pathway, where ^{˜}98% of the cells become exocrine, ductular, or matrix cells, and ^{˜}2% become endocrine cells.

As used herein, the term “somatic cell” refers to are any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body-apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro. Unless otherwise indicated the methods described herein can be performed both in vivo and in vitro.

As used herein, the term “adult cell” refers to a cell found throughout the body after embryonic development.

The term “endoderm cell” as used herein refers to a cell which is from one of the three primary germ cell layers in the very early embryo (the other two germ cell layers are the mesoderm and ectoderm). The endoderm is the innermost of the three layers. An endoderm cell differentiates to give rise first to the embryonic gut and then to the linings of respiratory and digestive tracts (e.g. the intestine), the liver and the pancreas.

The term “a cell of endoderm origin” as used herein refers to any cell which has developed or differentiated from an endoderm cell. For example, a cell of endoderm origin includes cells of the liver, lung, pancreas, thymus, intestine, stomach and thyroid. Without wishing to be bound by theory, liver and pancreas progenitors (also referred to as pancreatic progenitors) are develop from endoderm cells in the embryonic foregut. Shortly after their specification, liver and pancreas progenitors rapidly acquire markedly different cellular functions and regenerative capacities. These changes are elicited by inductive signals and genetic regulatory factors that are highly conserved among vertebrates.

The term “pancreatic progenitor” or “pancreatic precursor” are used interchangeably herein and refer to a stem cell which is capable of forming any of; pancreatic endocrine cells, or pancreatic exocrine cells or pancreatic duct cells. The term “pdx1-positive pancreatic progenitor” as used herein refers to a cell which is a pancreatic endoderm (PE) cell. A Pdx1-positive pancreatic progenitor expresses the marker Pdx1. Other markers include, but are not limited to Cdcp1, or Ptf1a, or HNF6 or NRx2.2. The expression of Pdx1 may be assessed by any method known by the skilled person such as immunochemistry using an anti-Pdx1 antibody or quantitative RT-PCR. The term “pdx1-positive, NKX6-1-positive pancreatic progenitor” as used herein refers to a cell which is a pancreatic endoderm (PE) cell. A pdx1-positive, NKX6-1-positive pancreatic progenitor expresses the markers Pdx1 and NKX6-1. Other markers include, but are not limited to Cdcp1, or Ptf1a, or HNF6 or NRx2.2. The expression of NKX6-1 may be assessed by any method known by the skilled person such as immunochemistry using an anti-NKX6-1 antibody or quantitative RT-PCR.

The terms “stem cell-derived p cell”, “SC-β cell”, and “mature SC-β cell” refer to cells (e.g., pancreatic β cells) that display at least one marker indicative of a pancreatic β cell, expresses insulin, and display a GSIS response characteristic of an endogenous mature β cell. In some embodiments, the “SC-β cell” comprises a mature pancreatic β cells. It is to be understood that the SC-β cells need not be derived (e.g., directly) from stem cells, as the methods of the disclosure are capable of deriving SC-β cells from any insulin-positive endocrine cell or precursor thereof using any cell as a starting point (e.g., one can use embryonic stem cells, induced-pluripotent stem cells, progenitor cells, partially reprogrammed somatic cells (e.g., a somatic cell which has been partially reprogrammed to an intermediate state between an induced pluripotent stem cell and the somatic cell from which it was derived), multipotent cells, totipotent cells, a transdifferentiated version of any of the foregoing cells, etc, as the invention is not intended to be limited in this manner). Examples of SC-β cells, and methods of obtaining such SC-β cells, are described in WO 2015/002724 and WO 2014/201167, both of which are incorporated herein by reference in their entirety.

The terms “stem cell-derived a cell”, “SC-α cell”, and “mature SC-α cell” refer to cells (e.g., pancreatic α cells) that display at least one marker indicative of a pancreatic α cell, express and secrete glucagon, and display an ultrastructure similar to cadaveric alpha cells. In some embodiments, the “SC-α cell” comprises a mature pancreatic α cell. It is to be understood that the SC-α cells need not be derived (e.g., directly) from stem cells, as the methods of the disclosure are capable of deriving SC-α cells from any insulin-positive endocrine cell or precursor thereof using any cell as a starting point (e.g., one can use embryonic stem cells, induced-pluripotent stem cells, progenitor cells, partially reprogrammed somatic cells (e.g., a somatic cell which has been partially reprogrammed to an intermediate state between an induced pluripotent stem cell and the somatic cell from which it was derived), multipotent cells, totipotent cells, a transdifferentiated version of any of the foregoing cells, etc, as the invention is not intended to be limited in this manner). Moreover it should be understood that an SC-α cell of the invention is a non-native, i.e., non-naturally occurring, non-endogenous, cell and has at least one characteristic that is different from a native/naturally-occurring/endogenous cell.

The term “exocrine cell” as used herein refers to a cell of an exocrine gland, i.e. a gland that discharges its secretion via a duct. In particular embodiments, an exocrine cell refers to a pancreatic exocrine cell, which is a pancreatic cell that produces enzymes that are secreted into the small intestine. These enzymes help digest food as it passes through the gastrointestinal tract. Pancreatic exocrine cells are also known as islets of Langerhans that secrete two hormones, insulin and glucagon.

The term “phenotype” refers to one or a number of total biological characteristics that define the cell or organism under a particular set of environmental conditions and factors, regardless of the actual genotype.

The term “pluripotent” as used herein refers to a cell with the capacity, under different conditions, to differentiate to more than one differentiated cell type, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g. iPS cells as that term is defined herein) also have the characteristic of the capacity of extended passaging without loss of growth potential, relative to primary cell parents, which generally have capacity for only a limited number of divisions in culture.

As used herein, the terms “iPS cell” and “induced pluripotent stem cell” are used interchangeably and refers to a pluripotent stem cell artificially derived (e.g., induced or by complete reversal) from a non-pluripotent cell, typically an adult somatic cell, for example, by inducing a forced expression of one or more genes.

The term “progenitor” or “precursor” cell are used interchangeably herein and refer to cells that have a cellular phenotype that is more primitive (i.e., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation” by persons of ordinary skill in the art. As used herein, the term “pluripotent stem cell” includes embryonic stem cells, induced pluripotent stem cells, placental stem cells, etc.

In the context of cell ontogeny, the adjective “differentiated”, or “differentiating” is a relative term meaning a “differentiated cell” is a cell that has progressed further down the developmental pathway than the cell it is being compared with. Thus, stem cells can differentiate to lineage-restricted precursor cells (such as a mesodermal stem cell), which in turn can differentiate into other types of precursor cells further down the pathway (such as an cardiomyocyte precursor), and then to an end-stage differentiated cell, which plays a characteristic role in a certain tissue type, and may or may not retain the capacity to proliferate further.

The term “embryonic stem cell” is used to refer to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” or “ASC” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells. As indicated above, stem cells have been found resident in virtually every tissue. Accordingly, the present invention appreciates that stem cell populations can be isolated from virtually any animal tissue.

The term “reprogramming” as used herein refers to the process that alters or reverses the differentiation state of a somatic cell. The cell can either be partially or terminally differentiated prior to the reprogramming. Reprogramming encompasses complete reversion of the differentiation state of a somatic cell to a pluripotent cell. Such complete reversal of differentiation produces an induced pluripotent (iPS) cell. Reprogramming as used herein also encompasses partial reversion of a cells differentiation state, for example to a multipotent state or to a somatic cell that is neither pluripotent or multipotent, but is a cell that has lost one or more specific characteristics of the differentiated cell from which it arises, e.g. direct reprogramming of a differentiated cell to a different somatic cell type. Reprogramming generally involves alteration, e.g., reversal, of at least some of the heritable patterns of nucleic acid modification (e.g., methylation), chromatin condensation, epigenetic changes, genomic imprinting, etc., that occur during cellular differentiation as a zygote develops into an adult.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

As used herein, the term “contacting” (i.e., contacting at least one endocrine cell or a precursor thereof with a maturation factor, or combination of maturation factors) is intended to include incubating the maturation factor and the cell together in vitro (e.g., adding the maturation factors to cells in culture). In some embodiments, the term “contacting” is not intended to include the in vivo exposure of cells to the compounds as disclosed herein that may occur naturally in a subject (i.e., exposure that may occur as a result of a natural physiological process). The step of contacting at least one endocrine cell or a precursor thereof with a maturation factor as in the embodiments described herein can be conducted in any suitable manner. For example, the cells may be treated in adherent culture, or in suspension culture. In some embodiments, the cells are treated in conditions that promote cell clustering. The disclosure contemplates any conditions which promote cell clustering. Examples of conditions that promote cell clustering include, without limitation, suspension culture in low attachment tissue culture plates, spinner flasks, aggrewell plates. In some embodiments, the inventors have observed that clusters have remained stable in media containing 10% serum. In some embodiments, the conditions that promote clustering include a low serum medium.

It is understood that the cells contacted with a maturation factor can also be simultaneously or subsequently contacted with another agent, such as a growth factor or other differentiation agent or environments to stabilize the cells, or to differentiate the cells further.

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

The term “cell line” refers to a population of largely or substantially identical cells that has typically been derived from a single ancestor cell or from a defined and/or substantially identical population of ancestor cells. The cell line may have been or may be capable of being maintained in culture for an extended period (e.g., months, years, for an unlimited period of time). It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other. In some embodiments, a cell line comprises a stem cell derived cell described herein.

The term “exogenous” refers to a substance present in a cell or organism other than its native source. For example, the terms “exogenous nucleic acid” or “exogenous protein” refer to a nucleic acid or protein that has been introduced by a process involving the hand of man into a biological system such as a cell or organism in which it is not normally found or in which it is found in lower amounts. A substance will be considered exogenous if it is introduced into a cell or an ancestor of the cell that inherits the substance. In contrast, the term “endogenous” refers to a substance that is native to the biological system.

The term “expression” refers to the cellular processes involved in producing RNA and proteins and as appropriate, secreting proteins, including where applicable, but not limited to, for example, transcription, translation, folding, modification and processing. “Expression products” include RNA transcribed from a gene and polypeptides obtained by translation of mRNA transcribed from a gene.

The terms “genetically modified” or “engineered” cell as used herein refers to a cell into which an exogenous nucleic acid has been introduced by a process involving the hand of man (or a descendant of such a cell that has inherited at least a portion of the nucleic acid). The nucleic acid may for example contain a sequence that is exogenous to the cell, it may contain native sequences (i.e., sequences naturally found in the cells) but in a non-naturally occurring arrangement (e.g., a coding region linked to a promoter from a different gene), or altered versions of native sequences, etc. The process of transferring the nucleic acid into the cell can be achieved by any suitable technique. Suitable techniques include calcium phosphate or lipid-mediated transfection, electroporation, and transduction or infection using a viral vector. In some embodiments the polynucleotide or a portion thereof is integrated into the genome of the cell. The nucleic acid may have subsequently been removed or excised from the genome, provided that such removal or excision results in a detectable alteration in the cell relative to an unmodified but otherwise equivalent cell. It should be appreciated that the term genetically modified is intended to include the introduction of a modified RNA directly into a cell (e.g., a synthetic, modified RNA). Such synthetic modified RNAs include modifications to prevent rapid degradation by endo- and exo-nucleases and to avoid or reduce the cell's innate immune or interferon response to the RNA. Modifications include, but are not limited to, for example, (a) end modifications, e.g., 5′ end modifications (phosphorylation dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages. To the extent that such modifications interfere with translation (i.e., results in a reduction of 50% or more in translation relative to the lack of the modification—e.g., in a rabbit reticulocyte in vitro translation assay), the modification is not suitable for the methods and compositions described herein.

The term “identity” as used herein refers to the extent to which the sequence of two or more nucleic acids or polypeptides is the same. The percent identity between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. When computing the number of identical residues needed to achieve a particular percent identity, fractions are to be rounded to the nearest whole number. Percent identity can be calculated with the use of a variety of computer programs known in the art. For example, computer programs such as BLAST2, BLASTN, BLASTP, Gapped BLAST, etc., generate alignments and provide percent identity between sequences of interest. The algorithm of Karlin and Altschul (Karlin and Altschul, Proc. Natl. Acad. Sci. USA 87:22264-2268, 1990) modified as in Karlin and Altschul, Proc. Natl. Acad. ScL USA 90:5873-5877, 1993 is incorporated into the NBLAST and XBLAST programs of Altschul et al. (Altschul, et al., J. MoI. Biol. 215:403-410, 1990). To obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. A PAM250 or BLOSUM62 matrix may be used. Software for performing BLAST analyses is publicly available through the National Center for Biotechnology Information (NCBI). See the Web site having URL world-wide web address of: “ncbi.nlm nih.gov” for these programs. In a specific embodiment, percent identity is calculated using BLAST2 with default parameters as provided by the NCBI.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from.

The term “substantially pure”, with respect to a particular cell population, refers to a population of cells that is at least about 75%, preferably at least about 85%, more preferably at least about 90%, and most preferably at least about 95% pure, with respect to the cells making up a total cell population.

The terms “enriching” or “enriched” are used interchangeably herein and mean that the yield (fraction) of cells of one type is increased by at least 10% over the fraction of cells of that type in the starting culture or preparation.

The terms “renewal” or “self-renewal” or “proliferation” are used interchangeably herein, are used to refer to the ability of stem cells to renew themselves by dividing into the same non-specialized cell type over long periods, and/or many months to years. In some instances, proliferation refers to the expansion of cells by the repeated division of single cells into two identical daughter cells.

The term “lineages” as used herein describes a cell with a common ancestry or cells with a common developmental fate. For example, in the context of a cell that is of endoderm origin or is “endodermal linage” this means the cell was derived from an endoderm cell and can differentiate along the endoderm lineage restricted pathways, such as one or more developmental lineage pathways which give rise to definitive endoderm cells, which in turn can differentiate into liver cells, thymus, pancreas, lung and intestine.

As used herein, the term “xenogeneic” refers to cells that are derived from different species.

A “marker” as used herein is used to describe the characteristics and/or phenotype of a cell. Markers can be used for selection of cells comprising characteristics of interests. Markers will vary with specific cells. Markers are characteristics, whether morphological, functional or biochemical (enzymatic) characteristics of the cell of a particular cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers may be detected by any method available to one of skill in the art. Markers can also be the absence of a morphological characteristic or absence of proteins, lipids etc. Markers can be a combination of a panel of unique characteristics of the presence and absence of polypeptides and other morphological characteristics.

The term “modulate” is used consistently with its use in the art, i.e., meaning to cause or facilitate a qualitative or quantitative change, alteration, or modification in a process, pathway, or phenomenon of interest. Without limitation, such change may be an increase, decrease, or change in relative strength or activity of different components or branches of the process, pathway, or phenomenon. A “modulator” is an agent that causes or facilitates a qualitative or quantitative change, alteration, or modification in a process, pathway, or phenomenon of interest.

As used herein, the term “DNA” is defined as deoxyribonucleic acid.

The term “polynucleotide” is used herein interchangeably with “nucleic acid” to indicate a polymer of nucleosides. Typically a polynucleotide of this invention is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated.

The terms “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a non-polypeptide moiety covalently or non-covalently associated therewith is still considered a “polypeptide”. Exemplary modifications include glycosylation and palmitoylation. Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

The term a “variant” in referring to a polypeptide could be, e.g., a polypeptide at least 80%, 85%, 90%, 95%, 98%, or 99% identical to full length polypeptide. The variant could be a fragment of full length polypeptide. The variant could be a naturally occurring splice variant. The variant could be a polypeptide at least 80%, 85%, 90%, 95%, 98%, or 99% identical to a fragment of the polypeptide, wherein the fragment is at least 50%, 60%, 70%, 80%, 85%, 90%, 95%, 98%, or 99% as long as the full length wild type polypeptide or a domain thereof having an activity of interest. In some embodiments the domain is at least 100, 200, 300, or 400 amino acids in length, beginning at any amino acid position in the sequence and extending toward the C-terminus. Variations known in the art to eliminate or substantially reduce the activity of the protein are preferably avoided. In some embodiments, the variant lacks an N- and/or C-terminal portion of the full length polypeptide, e.g., up to 10, 20, or 50 amino acids from either terminus is lacking. In some embodiments the polypeptide has the sequence of a mature (full length) polypeptide, by which is meant a polypeptide that has had one or more portions such as a signal peptide removed during normal intracellular proteolytic processing (e.g., during co-translational or post-translational processing). In some embodiments wherein the protein is produced other than by purifying it from cells that naturally express it, the protein is a chimeric polypeptide, by which is meant that it contains portions from two or more different species. In some embodiments wherein a protein is produced other than by purifying it from cells that naturally express it, the protein is a derivative, by which is meant that the protein comprises additional sequences not related to the protein so long as those sequences do not substantially reduce the biological activity of the protein.

The term “functional fragments” as used herein is a polypeptide having amino acid sequence which is smaller in size than, but substantially homologous to the polypeptide it is a fragment of, and where the functional fragment polypeptide sequence is about at least 50%, or 60% or 70% or at 80% or 90% or 100% or greater than 100%, for example 1.5-fold, 2-fold, 3-fold, 4-fold or greater than 4-fold effective biological action as the polypeptide from which it is a fragment of Functional fragment polypeptides may have additional functions that can include decreased antigenicity, increased DNA binding (as in transcription factors), or altered RNA binding (as in regulating RNA stability or degradation).

The term “vector” refers to a carrier DNA molecule into which a DNA sequence can be inserted for introduction into a host cell. Preferred vectors are those capable of autonomous replication and/or expression of nucleic acids to which they are linked. Vectors capable of directing the expression of genes to which they are operatively linked are referred to herein as “expression vectors”. Thus, an “expression vector” is a specialized vector that contains the necessary regulatory regions needed for expression of a gene of interest in a host cell. In some embodiments the gene of interest is operably linked to another sequence in the vector. Vectors can be viral vectors or non-viral vectors. Should viral vectors be used, it is preferred the viral vectors are replication defective, which can be achieved for example by removing all viral nucleic acids that encode for replication. A replication defective viral vector will still retain its infective properties and enters the cells in a similar manner as a replicating adenoviral vector, however once admitted to the cell a replication defective viral vector does not reproduce or multiply. Vectors also encompass liposomes and nanoparticles and other means to deliver DNA molecule to a cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

The term “viral vectors” refers to the use of viruses, or virus-associated vectors as carriers of a nucleic acid construct into a cell. Constructs may be integrated and packaged into non-replicating, defective viral genomes like Adenovirus, Adeno-associated virus (AAV), or Herpes simplex virus (HSV) or others, including reteroviral and lentiviral vectors, for infection or transduction into cells. The vector may or may not be incorporated into the cell's genome. The constructs may include viral sequences for transfection, if desired. Alternatively, the construct may be incorporated into vectors capable of episomal replication, e.g EPV and EBV vectors.

The terms “regulatory sequence” and “promoter” are used interchangeably herein, and refer to nucleic acid sequences, such as initiation signals, enhancers, and promoters, which induce or control transcription of protein coding sequences with which they are operatively linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein. In some instances the promoter sequence is recognized by the synthetic machinery of the cell, or introduced synthetic machinery, required for initiating transcription of a specific gene.

As used herein, the term “transcription factor” refers to a protein that binds to specific parts of DNA using DNA binding domains and is part of the system that controls the transfer (or transcription) of genetic information from DNA to RNA. As used herein, “proliferating” and “proliferation” refer to an increase in the number of cells in a population (growth) by means of cell division. Cell proliferation is generally understood to result from the coordinated activation of multiple signal transduction pathways in response to the environment, including growth factors and other mitogens. Cell proliferation may also be promoted by release from the actions of intra- or extracellular signals and mechanisms that block or negatively affect cell proliferation.

The term “selectable marker” refers to a gene, RNA, or protein that when expressed, confers upon cells a selectable phenotype, such as resistance to a cytotoxic or cytostatic agent (e.g., antibiotic resistance), nutritional prototrophy, or expression of a particular protein that can be used as a basis to distinguish cells that express the protein from cells that do not. Proteins whose expression can be readily detected such as a fluorescent or luminescent protein or an enzyme that acts on a substrate to produce a colored, fluorescent, or luminescent substance (“detectable markers”) constitute a subset of selectable markers. The presence of a selectable marker linked to expression control elements native to a gene that is normally expressed selectively or exclusively in pluripotent cells makes it possible to identify and select somatic cells that have been reprogrammed to a pluripotent state. A variety of selectable marker genes can be used, such as neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also of use. As will be evident to one of skill in the art, the term “selectable marker” as used herein can refer to a gene or to an expression product of the gene, e.g., an encoded protein.

In some embodiments the selectable marker confers a proliferation and/or survival advantage on cells that express it relative to cells that do not express it or that express it at significantly lower levels. Such proliferation and/or survival advantage typically occurs when the cells are maintained under certain conditions, i.e., “selective conditions.” To ensure an effective selection, a population of cells can be maintained under conditions and for a sufficient period of time such that cells that do not express the marker do not proliferate and/or do not survive and are eliminated from the population or their number is reduced to only a very small fraction of the population. The process of selecting cells that express a marker that confers a proliferation and/or survival advantage by maintaining a population of cells under selective conditions so as to largely or completely eliminate cells that do not express the marker is referred to herein as “positive selection”, and the marker is said to be “useful for positive selection”. Negative selection and markers useful for negative selection are also of interest in certain of the methods described herein. Expression of such markers confers a proliferation and/or survival disadvantage on cells that express the marker relative to cells that do not express the marker or express it at significantly lower levels (or, considered another way, cells that do not express the marker have a proliferation and/or survival advantage relative to cells that express the marker). Cells that express the marker can therefore be largely or completely eliminated from a population of cells when maintained in selective conditions for a sufficient period of time.

A “reporter gene” as used herein encompasses any gene that is genetically introduced into a cell that adds to the phenotype of the stem cell. Reporter genes as disclosed in this invention are intended to encompass fluorescent, luminescent, enzymatic and resistance genes, but also other genes which can easily be detected by persons of ordinary skill in the art. In some embodiments of the invention, reporter genes are used as markers for the identification of particular stem cells, cardiovascular stem cells and their differentiated progeny. A reporter gene is generally operatively linked to sequences that regulate its expression in a manner dependent upon one or more conditions which are monitored by measuring expression of the reporter gene. In some cases, expression of the reporter gene may be determined in live cells. Where live cell reporter gene assays are used, reporter gene expression may be monitored at multiple time points, e.g., 2, 3, 4, 5, 6, 8, or 10 or more time points. In some cases, where a live cell reporter assay is used, reporter gene expression is monitored with a frequency of at least about 10 minutes to about 24 hours, e.g., 20 minutes, 1 hour, 2 hours, 3 hours, 4 hours, 5 hours, 6 hours, 7 hours, 8 hours, 9 hours, 10 hours, 12 hours, 18 hours, or another frequency from any integer between about 10 minutes to about 24 hours.

The terms “subject” and “individual” are used interchangeably herein, and refer to an animal, for example, a human from whom cells can be obtained and/or to whom treatment, including prophylactic treatment, with the cells as described herein, is provided. For treatment of those infections, conditions or disease states which are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

The terms “treat”, “treating”, “treatment”, etc., as applied to an isolated cell, include subjecting the cell to any kind of process or condition or performing any kind of manipulation or procedure on the cell. As applied to a subject, the terms refer to providing medical or surgical attention, care, or management to an individual. The individual is usually ill or injured, or at increased risk of becoming ill relative to an average member of the population and in need of such attention, care, or management.

As used herein, the term “treating” and “treatment” refers to administering to a subject an effective amount of a composition so that the subject as a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. “Treatment” can also mean prolonging survival as compared to expected survival if not receiving treatment.

As used herein, the terms “administering,” “introducing” and “transplanting” are used interchangeably in the context of the placement of cells of the invention into a subject, by a method or route which results in at least partial localization of the introduced cells at a desired site. The cells can be implanted directly to the pancreas or gastrointestinal tract, or alternatively be administered by any appropriate route which results in delivery to a desired location in the subject where at least a portion of the implanted cells or components of the cells remain viable. The period of viability of the cells after administration to a subject can be as short as a few hours, e.g. twenty-four hours, to a few days, to as long as several years. In some instances, the cells can also be administered subcutaneously, for example, in a capsule (e.g., microcapsule) to maintain the implanted cells at the implant location and avoid migration of the implanted cells.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration,” “administered systemically”, “peripheral administration” and “administered peripherally” as used herein mean the administration of cardiovascular stem cells and/or their progeny and/or compound and/or other material other than directly into the central nervous system, such that it enters the animal's system and, thus, is subject to metabolism and other like processes, for example, subcutaneous administration.

The term “tissue” refers to a group or layer of specialized cells which together perform certain special functions. The term “tissue-specific” refers to a source of cells from a specific tissue.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

Stem cells are cells that retain the ability to renew themselves through mitotic cell division and can differentiate into a diverse range of specialized cell types. The two broad types of mammalian stem cells are: embryonic stem (ES) cells that are found in blastocysts, and adult stem cells that are found in adult tissues. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues. Pluripotent stem cells can differentiate into cells derived from any of the three germ layers.

While certain embodiments are described below in reference to the use of stem cells, germ cells may be used in place of, or with, the stem cells to provide at least one differentiated cell, using similar protocols as the illustrative protocols described herein. Suitable germ cells can be prepared, for example, from primordial germ cells present in human fetal material taken about 8-11 weeks after the last menstrual period. Illustrative germ cell preparation methods are described, for example, in Shamblott et al., Proc. Natl. Acad. Sci. USA 95:13726, 1998 and U.S. Pat. No. 6,090,622.

ES cells, e.g., human embryonic stem cells (hESCs) or mouse embryonic stem cells (mESCs), with a virtually endless replication capacity and the potential to differentiate into most cell types, present, in principle, an unlimited starting material to generate the differentiated cells for clinical therapy (stemcells.nih.gov/info/scireport/2006report.htm, 2006).

hESC cells, are described, for example, by Cowan et al. (N Engl. J. Med. 350:1353, 2004) and Thomson et al. (Science 282:1145, 1998); embryonic stem cells from other primates, Rhesus stem cells (Thomson et al., Proc. Natl. Acad. Sci. USA 92:7844, 1995), marmoset stem cells (Thomson et al., Biol. Reprod. 55:254, 1996) and human embryonic germ (hEG) cells (Shamblott et al., Proc. Natl. Acad. Sci. USA 95:13726, 1998) may also be used in the methods disclosed herein. mESCs, are described, for example, by Tremml et al. (Curr Protoc Stem Cell Biol. Chapter 1: Unit 1C.4, 2008). The stem cells may be, for example, unipotent, totipotent, multipotent, or pluripotent. In some examples, any cells of primate origin that are capable of producing progeny that are derivatives of at least one germinal layer, or all three germinal layers, may be used in the methods disclosed herein.

In certain examples, ES cells may be isolated, for example, as described in Cowan et al. (N Engl. J. Med. 350:1353, 2004) and U.S. Pat. No. 5,843,780 and Thomson et al., Proc. Natl. Acad. Sci. USA 92:7844, 1995. For example, hESCs cells can be prepared from human blastocyst cells using the techniques described by Thomson et al. (U.S. Pat. No. 6,200,806; Science 282:1145, 1998; Curr. Top. Dev. Biol. 38:133 ff., 1998) and Reubinoff et al, Nature Biotech. 18:399, 2000. Equivalent cell types to hESCs include their pluripotent derivatives, such as primitive ectoderm-like (EPL) cells, as outlined, for example, in WO 01/51610 (Bresagen). hESCs can also be obtained from human pre-implantation embryos. Alternatively, in vitro fertilized (IVF) embryos can be used, or one-cell human embryos can be expanded to the blastocyst stage (Bongso et al., Hum Reprod 4: 706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses can be isolated by immunosurgery, in which blastocysts are exposed to a 1:50 dilution of rabbit anti-human spleen cell antiserum for 30 min, then washed for 5 min three times in DMEM, and exposed to a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers. After 9 to 15 days, inner cell mass-derived outgrowths can be dissociated into clumps, either by exposure to calcium and magnesium-free phosphate-buffered saline (PBS) with 1 mM EDTA, by exposure to dispase or trypsin, or by mechanical dissociation with a micropipette; and then replated on mEF in fresh medium. Growing colonies having undifferentiated morphology can be individually selected by micropipette, mechanically dissociated into clumps, and replated. ES-like morphology is characterized as compact colonies with apparently high nucleus to cytoplasm ratio and prominent nucleoli. Resulting hESCs can then be routinely split every 1-2 weeks, for example, by brief trypsinization, exposure to Dulbecco's PBS (containing 2 mM EDTA), exposure to type IV collagenase (about 200 U/mL; Gibco) or by selection of individual colonies by micropipette. In some examples, clump sizes of about 50 to 100 cells are optimal. mESCs cells can be prepared from using the techniques described by e.g., Conner et al. (Curr. Prot. in Mol. Biol. Unit 23.4, 2003).

Embryonic stem cells can be isolated from blastocysts of members of the primate species (U.S. Pat. No. 5,843,780; Thomson et al., Proc. Natl. Acad. Sci. USA 92:7844, 1995). Human embryonic stem (hES) cells can be prepared from human blastocyst cells using the techniques described by Thomson et al. (U.S. Pat. No. 6,200,806; Science 282:1145, 1998; Curr. Top. Dev. Biol. 38:133 ff., 1998) and Reubinoff et al, Nature Biotech. 18:399, 2000. Equivalent cell types to hES cells include their pluripotent derivatives, such as primitive ectoderm-like (EPL) cells, as outlined in WO 01/51610 (Bresagen).

Alternatively, in some embodiments, hES cells can be obtained from human preimplantation embryos. Alternatively, in vitro fertilized (IVF) embryos can be used, or one-cell human embryos can be expanded to the blastocyst stage (Bongso et al., Hum Reprod 4: 706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses are isolated by immunosurgery, in which blastocysts are exposed to a 1:50 dilution of rabbit anti-human spleen cell antiserum for 30 min, then washed for 5 min three times in DMEM, and exposed to a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers.

After 9 to 15 days, inner cell mass-derived outgrowths are dissociated into clumps, either by exposure to calcium and magnesium-free phosphate-buffered saline (PBS) with 1 mM EDTA, by exposure to dispase or trypsin, or by mechanical dissociation with a micropipette; and then replated on mEF in fresh medium. Growing colonies having undifferentiated morphology are individually selected by micropipette, mechanically dissociated into clumps, and replated. ES-like morphology is characterized as compact colonies with apparently high nucleus to cytoplasm ratio and prominent nucleoli. Resulting ES cells are then routinely split every 1-2 weeks by brief trypsinization, exposure to Dulbecco's PBS (containing 2 mM EDTA), exposure to type IV collagenase (^{˜}200 U/mL; Gibco) or by selection of individual colonies by micropipette. Clump sizes of about 50 to 100 cells are optimal.

In some embodiments, human Embryonic Germ (hEG) cells are pluripotent stem cells which can be used in the methods as disclosed herein to differentiate into primitive endoderm cells. hEG cells can be used be prepared from primordial germ cells present in human fetal material taken about 8-11 weeks after the last menstrual period. Suitable preparation methods are described in Shamblott et al., Proc. Natl. Acad. Sci. USA 95:13726, 1998 and U.S. Pat. No. 6,090,622, which is incorporated herein in its entirety by reference.

Briefly, genital ridges processed to form disaggregated cells. EG growth medium is DMEM, 4500 mg/L D-glucose, 2200 mg/L mM NaHCO₃; 15% ES qualified fetal calf serum (BRL); 2 mM glutamine (BRL); 1 mM sodium pyruvate (BRL); 1000-2000 U/mL human recombinant leukemia inhibitory factor (LIF, Genzyme); 1-2 ng/mL human recombinant bFGF (Genzyme); and 10 μM forskolin (in 10% DMSO). Ninety-six well tissue culture plates are prepared with a sub-confluent layer of feeder cells (e.g., STO cells, ATCC No. CRL 1503) cultured for 3 days in modified EG growth medium free of LIF, bFGF or forskolin, inactivated with 5000 rad γ-irradiation ^{˜}0.2 mL of primary germ cell (PGC) suspension is added to each of the wells. The first passage is done after 7-10 days in EG growth medium, transferring each well to one well of a 24-well culture dish previously prepared with irradiated STO mouse fibroblasts. The cells are cultured with daily replacement of medium until cell morphology consistent with EG cells is observed, typically after 7-30 days or 1-4 passages.

In certain examples, the stem cells can be undifferentiated (e.g. a cell not committed to a specific linage) prior to exposure to at least one maturation factor according to the methods as disclosed herein, whereas in other examples it may be desirable to differentiate the stem cells to one or more intermediate cell types prior to exposure of the at least one maturation factor (s) described herein. For example, the stems cells may display morphological, biological or physical characteristics of undifferentiated cells that can be used to distinguish them from differentiated cells of embryo or adult origin. In some examples, undifferentiated cells may appear in the two dimensions of a microscopic view in colonies of cells with high nuclear/cytoplasmic ratios and prominent nucleoli. The stem cells may be themselves (for example, without substantially any undifferentiated cells being present) or may be used in the presence of differentiated cells. In certain examples, the stem cells may be cultured in the presence of suitable nutrients and optionally other cells such that the stem cells can grow and optionally differentiate. For example, embryonic fibroblasts or fibroblast-like cells may be present in the culture to assist in the growth of the stem cells. The fibroblast may be present during one stage of stem cell growth but not necessarily at all stages. For example, the fibroblast may be added to stem cell cultures in a first culturing stage and not added to the stem cell cultures in one or more subsequent culturing stages.

Stem cells used in all aspects of the present invention can be any cells derived from any kind of tissue (for example embryonic tissue such as fetal or pre-fetal tissue, or adult tissue), which stem cells have the characteristic of being capable under appropriate conditions of producing progeny of different cell types, e.g. derivatives of all of at least one of the 3 germinal layers (endoderm, mesoderm, and ectoderm). These cell types may be provided in the form of an established cell line, or they may be obtained directly from primary embryonic tissue and used immediately for differentiation. Included are cells listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)). In some embodiments, the source of human stem cells or pluripotent stem cells used for chemically-induced differentiation into mature, insulin positive cells did not involve destroying a human embryo.

In another embodiment, the stem cells can be isolated from tissue including solid tissue. In some embodiments, the tissue is skin, fat tissue (e.g. adipose tissue), muscle tissue, heart or cardiac tissue. In other embodiments, the tissue is for example but not limited to, umbilical cord blood, placenta, bone marrow, or chondral.

Stem cells of interest also include embryonic cells of various types, exemplified by human embryonic stem (hES) cells, described by Thomson et al. (1998) Science 282:1145; embryonic stem cells from other primates, such as Rhesus stem cells (Thomson et al. (1995) Proc. Natl. Acad. Sci. USA 92:7844); marmoset stem cells (Thomson et al. (1996) Biol. Reprod. 55:254); and human embryonic germ (hEG) cells (Shambloft et al., Proc. Natl. Acad. Sci. USA 95:13726, 1998). Also of interest are lineage committed stem cells, such as mesodermal stem cells and other early cardiogenic cells (see Reyes et al. (2001) Blood 98:2615-2625; Eisenberg & Bader (1996) Circ Res. 78(2):205-16; etc.) The stem cells may be obtained from any mammalian species, e.g. human, equine, bovine, porcine, canine, feline, rodent, e.g. mice, rats, hamster, primate, etc. In some embodiments, a human embryo was not destroyed for the source of pluripotent cell used on the methods and compositions as disclosed herein.

ES cells are considered to be undifferentiated when they have not committed to a specific differentiation lineage. Such cells display morphological characteristics that distinguish them from differentiated cells of embryo or adult origin. Undifferentiated ES cells are easily recognized by those skilled in the art, and typically appear in the two dimensions of a microscopic view in colonies of cells with high nuclear/cytoplasmic ratios and prominent nucleoli. Undifferentiated ES cells express genes that may be used as markers to detect the presence of undifferentiated cells, and whose polypeptide products may be used as markers for negative selection. For example, see U.S. application Ser. No. 2003/0224411 A1; Bhattacharya (2004) Blood 103(8):2956-64; and Thomson (1998), supra., each herein incorporated by reference. Human ES cell lines express cell surface markers that characterize undifferentiated nonhuman primate ES and human EC cells, including stage-specific embryonic antigen (SSEA)-3, SSEA-4, TRA-1-60, TRA-1-81, and alkaline phosphatase. The globo-series glycolipid GL7, which carries the SSEA-4 epitope, is formed by the addition of sialic acid to the globo-series glycolipid GbS, which carries the SSEA-3 epitope. Thus, GL7 reacts with antibodies to both SSEA-3 and SSEA-4. The undifferentiated human ES cell lines did not stain for SSEA-1, but differentiated cells stained strongly for SSEA-I. Methods for proliferating hES cells in the undifferentiated form are described in WO 99/20741, WO 01/51616, and WO 03/020920.

A mixture of cells from a suitable source of endothelial, muscle, and/or neural stem cells can be harvested from a mammalian donor by methods known in the art. A suitable source is the hematopoietic microenvironment. For example, circulating peripheral blood, preferably mobilized (i.e., recruited), may be removed from a subject. Alternatively, bone marrow may be obtained from a mammal, such as a human patient, undergoing an autologous transplant. In some embodiments, stem cells can be obtained from the subjects adipose tissue, for example using the CELUTION™ SYSTEM from Cytori, as disclosed in U.S. Pat. Nos. 7,390,484 and 7,429,488 which is incorporated herein in its entirety by reference.

In some embodiments, human umbilical cord blood cells (HUCBC) are useful in the methods as disclosed herein. Human UBC cells are recognized as a rich source of hematopoietic and mesenchymal progenitor cells (Broxmeyer et al., 1992 Proc. Natl. Acad. Sci. USA 89:4109-4113). Previously, umbilical cord and placental blood were considered a waste product normally discarded at the birth of an infant. Cord blood cells are used as a source of transplantable stem and progenitor cells and as a source of marrow repopulating cells for the treatment of malignant diseases (i.e. acute lymphoid leukemia, acute myeloid leukemia, chronic myeloid leukemia, myelodysplastic syndrome, and nueroblastoma) and non-malignant diseases such as Fanconi's anemia and aplastic anemia (Kohli-Kumar et al., 1993 Br. J. Haematol. 85:419-422; Wagner et al., 1992 Blood 79; 1874-1881; Lu et al., 1996 Crit. Rev. Oncol. Hematol 22:61-78; Lu et al., 1995 Cell Transplantation 4:493-503). A distinct advantage of HUCBC is the immature immunity of these cells that is very similar to fetal cells, which significantly reduces the risk for rejection by the host (Taylor & Bryson, 1985J. Immunol. 134:1493-1497). Human umbilical cord blood contains mesenchymal and hematopoietic progenitor cells, and endothelial cell precursors that can be expanded in tissue culture (Broxmeyer et al., 1992 Proc. Natl. Acad. Sci. USA 89:4109-4113; Kohli-Kumar et al., 1993 Br. J. Haematol. 85:419-422; Wagner et al., 1992 Blood 79; 1874-1881; Lu et al., 1996 Crit. Rev. Oncol. Hematol 22:61-78; Lu et al., 1995 Cell Transplantation 4:493-503; Taylor & Bryson, 1985J. Immunol. 134:1493-1497 Broxmeyer, 1995 Transfusion 35:694-702; Chen et al., 2001 Stroke 32:2682-2688; Nieda et al., 1997 Br. J. Haematology 98:775-777; Erices et al., 2000 Br. J. Haematology 109:235-242). The total content of hematopoietic progenitor cells in umbilical cord blood equals or exceeds bone marrow, and in addition, the highly proliferative hematopoietic cells are eightfold higher in HUCBC than in bone marrow and express hematopoietic markers such as CD14, CD34, and CD45 (Sanchez-Ramos et al., 2001 Exp. Neur. 171:109-115; Bicknese et al., 2002 Cell Transplantation 11:261-264; Lu et al., 1993 J. Exp Med. 178:2089-2096).

In another embodiment, pluripotent cells are cells in the hematopoietic microenvironment, such as the circulating peripheral blood, preferably from the mononuclear fraction of peripheral blood, umbilical cord blood, bone marrow, fetal liver, or yolk sac of a mammal. The stem cells, especially neural stem cells, may also be derived from the central nervous system, including the meninges.

In another embodiment, pluripotent cells are present in embryoid bodies are formed by harvesting ES cells with brief protease digestion, and allowing small clumps of undifferentiated human ESCs to grow in suspension culture. Differentiation is induced by withdrawal of conditioned medium. The resulting embryoid bodies are plated onto semi-solid substrates. Formation of differentiated cells may be observed after around about 7 days to around about 4 weeks. Viable differentiating cells from in vitro cultures of stem cells are selected for by partially dissociating embryoid bodies or similar structures to provide cell aggregates. Aggregates comprising cells of interest are selected for phenotypic features using methods that substantially maintain the cell to cell contacts in the aggregate.

In an alternative embodiment, the stem cells can be reprogrammed stem cells, such as stem cells derived from somatic or differentiated cells. In such an embodiment, the de-differentiated stem cells can be for example, but not limited to, neoplastic cells, tumor cells and cancer cells or alternatively induced reprogrammed cells such as induced pluripotent stem cells or iPS cells.

Illustrative methods for molecular genetics and genetic engineering that may be used in the technology described herein may be found, for example, in current editions of Molecular Cloning: A Laboratory Manual, (Sambrook et al., Cold Spring Harbor); Gene Transfer Vectors for Mammalian Cells (Miller & Calos eds.); and Current Protocols in Molecular Biology (F. M. Ausubel et al. eds., Wiley & Sons). Cell biology, protein chemistry, and antibody techniques can be found, for example, in Current Protocols in Protein Science (J. E. Colligan et al. eds., Wiley & Sons); Current Protocols in Cell Biology (J. S. Bonifacino et al., Wiley & Sons) and Current protocols in Immunology (J. E. Colligan et al. eds., Wiley & Sons.). Illustrative reagents, cloning vectors, and kits for genetic manipulation may be commercially obtained, for example, from BioRad, Stratagene, Invitrogen, ClonTech, and Sigma-Aldrich Co.

Suitable cell culture methods may be found, for example, in Cell culture methods are described generally in the current edition of Culture of Animal Cells: A Manual of Basic Technique (R. I. Freshney ed., Wiley & Sons); General Techniques of Cell Culture (M. A. Harrison & I. F. Rae, Cambridge Univ. Press), and Embryonic Stem Cells: Methods and Protocols (K. Turksen ed., Humana Press). Suitable tissue culture supplies and reagents are commercially available, for example, from Gibco/BRL, Nalgene-Nunc International, Sigma Chemical Co., and ICN Biomedicals.

Pluripotent stem cells can be propagated by one of ordinary skill in the art and continuously in culture, using culture conditions that promote proliferation without promoting differentiation. Exemplary serum-containing ES medium is made with 80% DMEM (such as Knock-Out DMEM, Gibco), 20% of either defined fetal bovine serum (FBS, Hyclone) or serum replacement (WO 98/30679), 1% non-essential amino acids, 1 mM L-glutamine, and 0.1 mM β-mercaptoethanol. Just before use, human bFGF is added to 4 ng/mL (WO 99/20741, Geron Corp.). Traditionally, ES cells are cultured on a layer of feeder cells, typically fibroblasts derived from embryonic or fetal tissue.

Scientists at Geron have discovered that pluripotent SCs can be maintained in an undifferentiated state even without feeder cells. The environment for feeder-free cultures includes a suitable culture substrate, particularly an extracellular matrix such as Matrigel® or laminin. Typically, enzymatic digestion is halted before cells become completely dispersed (say, ˜5 min with collagenase IV). Clumps of ˜10 to 2,000 cells are then plated directly onto the substrate without further dispersal.

Feeder-free cultures are supported by a nutrient medium containing factors that support proliferation of the cells without differentiation. Such factors may be introduced into the medium by culturing the medium with cells secreting such factors, such as irradiated (˜4,000 rad) primary mouse embryonic fibroblasts, telomerized mouse fibroblasts, or fibroblast-like cells derived from pPS cells. Medium can be conditioned by plating the feeders at a density of ˜5-6×10⁴ cm⁻² in a serum free medium such as KO DMEM supplemented with 20% serum replacement and 4 ng/mL bFGF. Medium that has been conditioned for 1-2 days is supplemented with further bFGF, and used to support pluripotent SC culture for 1-2 days. Features of the feeder-free culture method are further discussed in International Patent Publication WO 01/51616; and Xu et al., Nat. Biotechnol. 19:971, 2001.

Under the microscope, ES cells appear with high nuclear/cytoplasmic ratios, prominent nucleoli, and compact colony formation with poorly discernable cell junctions. Primate ES cells express stage-specific embryonic antigens (SSEA) 3 and 4, and markers detectable using antibodies designated Tra-1-60 and Tra-1-81 (Thomson et al., Science 282:1145, 1998). Mouse ES cells can be used as a positive control for SSEA-1, and as a negative control for SSEA-4, Tra-1-60, and Tra-1-81. SSEA-4 is consistently present human embryonal carcinoma (hEC) cells. Differentiation of pluripotent SCs in vitro results in the loss of SSEA-4, Tra-1-60, and Tra-1-81 expression, and increased expression of SSEA-1, which is also found on undifferentiated hEG cells.

Aspects of the disclosure relate to generating stem cell-derived cells (e.g., SC-α cells). Generally, the at least one stem cell-derived cell or precursor thereof, e.g., polyhormonal cells produced according to the methods disclosed herein can comprise a mixture or combination of different cells, for example a mixture of cells such as endocrine progenitor cells, pancreatic progenitor cells, gut tube endoderm cells, definitive endoderm cells, and/or other pluripotent or stem cells.

The at least one stem cell-derived cell or precursor thereof can be produced according to any suitable culturing protocol to differentiate a stem cell or pluripotent cell to a desired stage of differentiation. In some embodiments, the at least one stem cell-derived cell or the precursor thereof are produced by culturing at least one pluripotent cell for a period of time and under conditions suitable for the at least one pluripotent cell to differentiate into the at least one stem cell-derived cell or the precursor thereof.

In some embodiments, the at least one stem cell-derived cell or precursor thereof is a substantially pure population of stem cell-derived cells or precursors thereof. In some embodiments, a population of stem cell-derived cells or precursors thereof comprises a mixture of pluripotent cells or differentiated cells (e.g., a mixture of SC-α cells and SC-β cells). In some embodiments, a population SC-α cells or precursors thereof are substantially free or devoid of embryonic stem cells or pluripotent cells or iPS cells.

In some embodiments, a somatic cell, e.g., fibroblast can be isolated from a subject, for example as a tissue biopsy, such as, for example, a skin biopsy, and reprogrammed into an induced pluripotent stem cell for further differentiation to produce the at least one stem cell-derived cell or precursor thereof for use in the compositions and methods described herein. In some embodiments, a somatic cell, e.g., fibroblast is maintained in culture by methods known by one of ordinary skill in the art, and in some embodiments, propagated prior to being converted into stem cell-derived cells by the methods as disclosed herein.

In some embodiments, the at least one stem cell-derived cell or precursor thereof is maintained in culture by methods known by one of ordinary skill in the art, and in some embodiments, propagated prior to being converted into stem cell-derived cells by the methods as disclosed herein.

Further, at least one stem cell-derived cell or precursor thereof, e.g., pancreatic progenitor can be from any mammalian species, with non-limiting examples including a murine, bovine, simian, porcine, equine, ovine, or human cell. For clarity and simplicity, the description of the methods herein refers to a mammalian stem cell-derived cell or precursor thereof but it should be understood that all of the methods described herein can be readily applied to other cell types of at least one stem cell-derived cell or precursor thereof. In some embodiments, the at least one stem cell-derived cell or precursor thereof is derived from a human individual.

In some embodiments stem cell-derived cells may be produced using the methods disclosed in WO 2015/002724 and WO 2014/201167, both of which are incorporated herein by reference. In other embodiments stem cell-derived cells (e.g., SC-α cells) are produced using an alpha cell differentiation protocol.

In certain embodiments a method for generating SC-α cells comprises contacting a cell population comprising progenitor cells (e.g., pancreatic progenitor cells or endocrine progenitor cells) directed to differentiate into SC-α cells, or cell precursors thereof, with an effective amount of agents or factors capable of directing the differentiation of such cells into functional SC-α cells. In some aspects the various stages of the differentiation protocol include embryonic stem cells (ESC), definitive endoderm cells (DE)), gut tube endoderm cells (GTE), pancreatic progenitor cells (PP), endocrine progenitor cells (EP), and polyhormonal cells (PH).

Definitive endoderm cells of use herein can be derived from any source or generated in accordance with any suitable protocol. In some aspects, pluripotent stem cells, e.g., iPSCs or hESCs, are differentiated to endoderm cells. In some aspects, the endoderm cells are further differentiated, e.g., to primitive gut tube cells, pancreatic progenitor cells, endocrine progenitor cells, or polyhormonal cells, followed by induction or maturation to SC-α cells.

In some embodiments definitive endoderm cells can be obtained by differentiating at least some pluripotent cells in a population into definitive endoderm cells, e.g., by contacting a population of pluripotent cells with i) at least one growth factor from the TGF-β superfamily, and ii) a WNT signaling pathway activator, to induce the differentiation of at least some of the pluripotent cells into definitive endoderm cells, wherein the definitive endoderm cells express at least one marker characteristic of definitive endoderm.

The disclosure contemplates the use of any growth factor from the TGF-β superfamily that induces the pluripotent stem cells to differentiate into definitive endoderm cells (e.g., alone, or in combination with a WNT signaling pathway activator). In some embodiments, the at least one growth factor from the TGF-β superfamily comprises Activin A. Other examples of growth factors from the TGF-β superfamily include GDF8 and GDF11, as well as agents that mimic the growth factors from the TGF-β superfamily (e.g., IDE1 and IDE2).

The disclosure contemplates the use of any WNT signaling pathway activator that induces the pluripotent stem cells to differentiate into definitive endoderm cells (e.g., alone, or in combination with a growth factor from the TGF-β superfamily). In some embodiments, the WNT signaling pathway activator comprises CHIR99021. Other examples of WNT signaling pathway activators include Wnt3a recombinant protein or a GSK3 inhibitor.

The skilled artisan will appreciate that the concentrations of agents (e.g., growth factors) employed may vary. In some embodiments, the pluripotent cells are contacted with the at least one growth factor from the TGF-β superfamily at a concentration of between 10 ng/mL-1000 ng/mL. In some embodiments, the pluripotent cells are contacted with the at least one growth factor from the TGF-β superfamily at a concentration of 100 ng/mL. In some embodiments, the pluripotent cells are contacted with the at least one growth factor from the TGF-β superfamily at a concentration of 20 ng/mL, 30 ng/mL, 40 ng/mL, 50 ng/mL, 60 ng/mL, 70 ng/mL, 80 ng/mL, or 90 ng/mL. In some embodiments, the pluripotent cells are contacted with the at least one growth factor from the TGF-β superfamily at a concentration of 91 ng/mL, 92 ng/mL, 93 ng/mL, 94 ng/mL, 95 ng/mL, 96 ng/mL, 97 ng/mL, 98 ng/mL or 99 ng/mL. In some embodiments, the pluripotent cells are contacted with the at least one growth factor from the TGF-β superfamily at a concentration of 110 ng/mL, 120 ng/mL, 130 ng/mL, 140 ng/mL, 150 ng/mL, 160 ng/mL, 170 ng/mL, 180 ng/mL, or 190 ng/mL. In some embodiments, the pluripotent cells are contacted with the at least one growth factor from the TGF-β superfamily at a concentration of 101 ng/mL, 102 ng/mL, 103 ng/mL, 104 ng/mL, 105 ng/mL, 106 ng/mL, 107 ng/mL, 108 ng/mL or 109 ng/mL.

In some embodiments the pluripotent cells are contacted with the WNT signaling pathway activator at a concentration of between 0.4 uM-14 uM. In some embodiments, the pluripotent cells are contacted with the WNT signaling pathway activator at a concentration of 3 uM. In some embodiments, the pluripotent cells are contacted with the WNT signaling pathway activator at a concentration of 0.5 uM, 1 uM, 1.5 uM or 2 uM. In some embodiments, the pluripotent cells are contacted with the WNT signaling pathway activator at a concentration of 3.5 uM, 4 uM, 4.5 uM, 5 uM, 5.5 uM, or 6 uM. In some embodiments, the pluripotent cells are contacted with the WNT signaling pathway activator at a concentration of 2.1 uM, 2.2 uM, 2.3 uM, 2.4 uM, 2.5 uM, 2.6 uM, 2.7 uM, 2.8 uM, or 2.9 uM. In some embodiments, the pluripotent cells are contacted with the WNT signaling pathway activator at a concentration of 3.1 uM, 3.2 uM, 3.3 uM, 3.4 uM, 3.5 uM, 3.6 uM, 3.7 uM, 3.8 uM, or 3.9 uM.

Generally, the pluripotent cells are maintained in suitable culture medium (e.g., suspension culture) for a period of time sufficient to induce the differentiation of at least some of the pluripotent cells into definitive endoderm cells. An exemplary suitable culture medium is shown in Table 1 below.

TABLE 1

| Agent | Amount | |
|---|---|---|
| MCDB131 | 1 | L |
| Glucose | 0.44 | |
| NaHCO3 | 2.46 | g |
| FAF-BSA | 20 | g |
| ITS-X | 20 | uL |
| Glutamax | 10 | mL |
| Vitamin C | 0.044 | g |
| Heparin | 0 | g |
| P/S | 10 | mL |

In some embodiments, a suitable culture medium for differentiating pluripotent cells into definitive endoderm cells comprises Si media.

In some embodiments, contacting the pluripotent cells is effected in suspension culture. In some embodiments, the suspension culture is maintained in a spinner flask. In some embodiments, the period of time is 3 days. In some embodiments, the at least one growth factor from the TGF-β superfamily, and WNT signaling pathway activator are added to the suspension culture on the first day. In some embodiments, the at least one growth factor from the TGF-β superfamily is replenished in the suspension culture on the second day. In some embodiments, the WNT signaling pathway activator is not replenished in the suspension culture on the second day. In some embodiments, the WNT signaling pathway activator is removed from the suspension culture on the second day. In some embodiments, the at least one growth factor from the TGF-β superfamily is replenished in the suspension culture on the second day, and the WNT signaling pathway activator is removed from the suspension culture or not replenished in the suspension culture on the second day. In some embodiments, neither the at least one growth factor from the TGF-β superfamily or the WNT signaling pathway activator are replenished in the suspension culture on the third day. In some embodiments, both the at least one growth factor from the TGF-β superfamily and the WNT signaling pathway activator are removed from the suspension culture on the third day.

The methods are capable of inducing the differentiation of at least one pluripotent cell in a population of cells into a definitive endoderm cell. Generally, any pluripotent cell can be differentiated into a definitive endoderm cell using a method described herein. In some embodiments, the pluripotent cells comprise induced pluripotent stem cells. In some embodiments, the pluripotent cells comprise embryonic stem cells. In some embodiments, the pluripotent cells comprise human cells.

In some embodiments, differentiating at least some pluripotent cells in a population into definitive endoderm cells is achieved by a process of contacting a population of pluripotent cells with i) Activin A and ii) CHIR99021, to induce the differentiation of at least some of the pluripotent cells in the population into definitive endoderm cells, wherein the definitive endoderm cells express at least one marker characteristic of definitive endoderm.

Primitive gut tube cells of use herein can be derived from any source or generated in accordance with any suitable protocol. In some aspects, definitive endoderm cells are differentiated to primitive gut tube cells. In some aspects, the primitive gut tube cells are further differentiated, e.g., to pancreatic progenitor cells, endocrine progenitor cells, polyhormonal cells, followed by induction or maturation to SC-α cells.

In some embodiments, primitive gut tube cells can be obtained by differentiating at least some definitive endoderm cells in a population into primitive gut tube cells, e.g., by contacting definitive endoderm cells with at least one growth factor from the fibroblast growth factor (FGF) family, to induce the differentiation of at least some of the definitive endoderm cells into primitive gut tube cells, wherein the primitive gut tube cells express at least one marker characteristic of primitive gut tube cells.

The disclosure contemplates the use of any growth factor from the FGF family that induces definitive endoderm cells to differentiate into primitive gut tube cells (e.g., alone, or in combination with other factors). In some embodiments, the at least one growth factor from the FGF family comprises keratinocyte growth factor (KGF). Other examples of growth factors from the FGF family include FGF2, FGF8B, FGF10, and FGF21.

The skilled artisan will appreciate that the concentrations of growth factor employed may vary. In some embodiments the definitive endoderm cells are contacted with the at least one growth factor from the FGF family at a concentration of between 5 ng/mL-500 ng/mL. In some embodiments the definitive endoderm cells are contacted with the at least one growth factor from the FGF family at a concentration of 50 ng/mL. In some embodiments the definitive endoderm cells are contacted with the at least one growth factor from the FGF family at a concentration of 10 ng/mL, 15 ng/mL, 20 ng/mL, 25 ng/mL, 30 ng/mL, 35 ng/mL, or 40 ng/mL. In some embodiments, the definitive endoderm cells are contacted with the at least one growth factor from the FGF family at a concentration of 60 ng/mL, 65 ng/mL, 70 ng/mL, 75 ng/mL, 80 ng/mL, 85 ng/mL, 90 ng/mL, 95 ng/mL or 100 ng/mL. In some embodiments the definitive endoderm cells are contacted with the at least one growth factor from the FGF family at a concentration of 41 ng/mL, 42 ng/mL, 43 ng/mL, 44 ng/mL, 45 ng/mL, 46 ng/mL, 47 ng/mL, 48 ng/mL or 49 ng/mL. In some embodiments the definitive endoderm cells are contacted with the at least one growth factor from the FGF family at a concentration of 51 ng/mL, 52 ng/mL, 53 ng/mL, 54 ng/mL, 55 ng/mL, 56 ng/mL, 57 ng/mL, 58 ng/mL or 59 ng/mL.

In some embodiments, the definitive endoderm cells are cultured in a suitable culture medium.

Generally, the definitive endoderm cells are maintained in a suitable culture medium (e.g., suspension culture) for a period of time sufficient to induce the differentiation of at least some of the definitive endoderm cells into primitive gut tube cells. An exemplary suitable culture medium is shown in Table 2 below.

TABLE 2

| Agent | Amount | |
|---|---|---|
| MCDB131 | 1 | L |
| Glucose | 0.44 | g |
| NaHCO3 | 1.23 | g |
| FAF-BSA | 20 | g |
| ITS-X | 20 | uL |
| Glutamax | 10 | mL |
| Vitamin C | 0.044 | g |
| Heparin | 0 | g |
| P/S | 10 | mL |

In some embodiments, a suitable culture medium for differentiating definitive endoderm cells into primitive gut tube cells comprises S2 media.

In some embodiments, contacting the definitive endoderm cells is effected in suspension culture. In some embodiments, the suspension culture is maintained in a spinner flask. In some embodiments, the period of time is between 2 days and 5 days. In some embodiments, the period of time is 2 days.

In some embodiments, definitive endoderm cells can be obtained by differentiating at least some of the definitive endoderm cells in a population into primitive gut tube cells, e.g., by contacting the definitive endoderm cells with KGF, to induce the differentiation of at least some of the definitive endoderm cells into primitive gut tube cells, wherein the primitive gut tube cells express at least one marker characteristic of primitive gut tube cells.

Pancreatic progenitor cells of use herein can be derived from any source or generated in accordance with any suitable protocol. In some aspects, primitive gut tube cells are differentiated to pancreatic progenitor cells. In some aspects, the pancreatic progenitor cells are further differentiated, e.g., endocrine progenitor cells, polyhormonal cells, followed by induction or maturation to SC-α cells.

In some aspects, pancreatic progenitor cells can be obtained by differentiating at least some primitive gut tube cells in a population into pancreatic progenitor cells, e.g., by contacting primitive gut tube cells with i) at least one bone morphogenic protein (BMP) signaling pathway inhibitor and ii) at least one retinoic acid (RA) signaling pathway activator, to induce the differentiation of at least some of the primitive gut tube cells into pancreatic progenitor cells, wherein the pancreatic progenitor cells express at least one marker characteristic of pancreatic progenitor cells.

The disclosure contemplates the use of any BMP signaling pathway inhibitor that induces primitive gut tube cells to differentiate into pancreatic progenitor cells (e.g., alone, or with at least one retinoic acid signaling pathway activator). In some embodiments, the BMP signaling pathway inhibitor comprises LDN193189. Other examples of BMP signaling pathway inhibitors include analogs or derivatives of LDN193189 (e.g., LDN13189 hydrochloride) and compounds of Formula I from U.S. Patent Publication No. 2011/0053930, incorporated herein by reference.

The disclosure contemplates the use of any RA signaling pathway activator that induces primitive gut tube cells to differentiate into pancreatic progenitor cells (e.g., alone, or with at least one BMP signaling pathway inhibitor). In some embodiments, the RA signaling pathway activator comprises retinoic acid. Other examples of RA signaling pathway activators include retinoic acid receptor agonists (e.g., CD 1530, AM 580, TTNPB, CD 437, Ch 55, BMS 961, AC 261066, AC 55649, AM 80, BMS 753, tazarotene, adapalene, and CD 2314).

The skilled artisan will appreciate that the concentrations of agents (e.g., growth factors) employed may vary. In some embodiments the primitive gut tube cells are contacted with the BMP signaling pathway inhibitor at a concentration of between 20 nM-2000 nM. In some embodiments the primitive gut tube cells are contacted with the BMP signaling pathway inhibitor at a concentration of 200 nM. In some embodiments the primitive gut tube cells are contacted with the BMP signaling pathway inhibitor at a concentration of 30 nM, 40 nM, 50 nM, 60 nM, 70 nM, 80 nM, 90 nM, 100 nM, 110 nM, 120 nM, 130 nM, 140 nM, 150 nM, 160 nM, 170 nM, 180 nM, or 190 nM. In some embodiments the primitive gut tube cells are contacted with the BMP signaling pathway inhibitor at a concentration of 191 nM, 192 nM, 193 nM, 194 nM, 195 nM, 196 nM, 197 nM, 198 nM, or 199 nM. In some embodiments the primitive gut tube cells are contacted with the BMP signaling pathway inhibitor at a concentration of 300 nM, 400 nM, 500 nM, 600 nM, 700 nM, 800 nM, 900 nM, 1000 nM, 1100 nM, 1200 nM, 1300 nM, 1400 nM, 1500 nM, 1600 nM, 1700 nM, 1800 nM, or 1900 nM. In some embodiments the primitive gut tube cells are contacted with the BMP signaling pathway inhibitor at a concentration of 210 nM, 220 nM, 230 nM, 240 nM, 250 nM, 260 nM, 270 nM, 280 nM, or 290 nM.

In some embodiments, the primitive gut tube cells are contacted with the RA signaling pathway activator at a concentration of between 0.01 uM-5.0 uM. In some embodiments, the primitive gut tube cells are contacted with the RA signaling pathway activator at a concentration of 2 uM. In some embodiments, the primitive gut tube cells are contacted with the RA signaling pathway activator at a concentration of 1.1 uM, 1.2 uM, 1.3 uM, 1.4 uM, 1.5 uM, 1.6 uM, 1.7 uM, 1.8 uM, or 1.9 uM. In some embodiments, the primitive gut tube cells are contacted with the RA signaling pathway activator at a concentration of 2.1 uM, 2.2 uM, 2.3 uM, 2.4 uM, 2.5 uM, 2.6 uM, 2.7 uM, 2.8 uM, or 2.9 uM.

Generally, the primitive gut tube cells are maintained in a suitable culture medium (e.g., suspension culture) for a period of time sufficient to induce the differentiation of at least some of the primitive gut tube cells into pancreatic progenitor cells. An exemplary suitable culture medium is shown in Table 3 below.

TABLE 3

| Agent | Amount | |
|---|---|---|
| MCDB131 | 1 | L |
| Glucose | 0.44 | g |
| NaHCO3 | 1.23 | |
| FAF-BSA | 20 | g |
| ITS-X | 5 | mL |
| Glutamax | 10 | mL |
| Vitamin C | 0.044 | g |
| Heparin | 0 | g |
| P/S | 10 | mL |

In some embodiments, S3 media can be used as a suitable culture medium for differentiating primitive gut tube cells into pancreatic progenitor cells.

In some embodiments, contacting the primitive gut tube cells is effected in suspension culture. In some embodiments, the suspension culture is maintained in a spinner flask. In some embodiments, the period of time is at least 2 days. In some embodiments, the suspension culture is replenished every day. In some embodiments, the at least one RA signaling pathway activator is added to the suspension culture on the first day of Stage 3 (e.g., on day 6 of the differentiation protocol). In some embodiments a BMP signaling pathway inhibitor is not added to the suspension culture during the first day of Stage 3. In some embodiments the at least one RA signaling pathway activator and BMP signaling pathway inhibitor are added to the suspension culture on the second day of Stage 3 (e.g., on day 7 of the differentiation protocol).

In some embodiments, pancreatic progenitor cells can be obtained by differentiating at least some of the primitive gut tube cells in a population into pancreatic progenitor cells, e.g., by contacting the primitive gut tube cells with i) LDN193189 and ii) RA, to induce the differentiation of at least some of the primitive gut tube cells into pancreatic progenitor cells, wherein the primitive gut tube cells express at least one marker characteristic of pancreatic progenitor cells. In some embodiments pancreatic progenitor cells are obtained by differentiating at least some of the primitive gut tube cells in a population into pancreatic progenitor cells, e.g., by contacting the primitive gut tube cells with LDN193189 for one day, and then contacting the cells with LDN193189 and RA for one day, to induce the differentiation of at least some of the primitive gut tube cells into pancreatic progenitor cells, wherein the pancreatic progenitor cells express at least one marker characteristic of pancreatic progenitor cells.

Endocrine progenitor cells of use herein can be derived from any source or generated in accordance with any suitable protocol. In some aspects, pancreatic progenitor cells are differentiated to endocrine progenitor cells. In some aspects, the endocrine progenitor cells are further differentiated to polyhormonal cells.

In some aspects, a method of producing an endocrine progenitor cell from an pancreatic progenitor cell comprises contacting a population of cells (e.g., under conditions that promote cell clustering) comprising pancreatic progenitor cells with at least one bone morphogenic protein (BMP) signaling pathway inhibitor to induce the differentiation of at least some of the pancreatic progenitor cells into endocrine progenitor cells, wherein the endocrine progenitor cells express at least one marker characteristic of endocrine progenitor cells.

The disclosure contemplates the use of any BMP signaling pathway inhibitor that induces pancreatic progenitor cells to differentiate into endocrine progenitor cells. In some embodiments, the BMP signaling pathway inhibitor comprises LDN193189.

The skilled artisan will appreciate that the concentrations of agents (e.g., growth factors) employed may vary. In some embodiments the pancreatic progenitor cells are contacted with the BMP signaling pathway inhibitor at a concentration of between 20 nM-2000 nM. In some embodiments the pancreatic progenitor cells are contacted with the BMP signaling pathway inhibitor at a concentration of 200 nM. In some embodiments the pancreatic progenitor cells are contacted with the BMP signaling pathway inhibitor at a concentration of 30 nM, 40 nM, 50 nM, 60 nM, 70 nM, 80 nM, 90 nM, 100 nM, 110 nM, 120 nM, 130 nM, 140 nM, 150 nM, 160 nM, 170 nM, 180 nM, or 190 nM. In some embodiments the pancreatic progenitor cells are contacted with the BMP signaling pathway inhibitor at a concentration of 191 nM, 192 nM, 193 nM, 194 nM, 195 nM, 196 nM, 197 nM, 198 nM, or 199 nM. In some embodiments the pancreatic progenitor cells are contacted with the BMP signaling pathway inhibitor at a concentration of 300 nM, 400 nM, 500 nM, 600 nM, 700 nM, 800 nM, 900 nM, 1000 nM, 1100 nM, 1200 nM, 1300 nM, 1400 nM, 1500 nM, 1600 nM, 1700 nM, 1800 nM, or 1900 nM. In some embodiments the pancreatic progenitor cells are contacted with the BMP signaling pathway inhibitor at a concentration of 210 nM, 220 nM, 230 nM, 240 nM, 250 nM, 260 nM, 270 nM, 280 nM, or 290 nM.

Generally, the pancreatic progenitor cells are maintained in a suitable culture medium (e.g., suspension culture) for a period of time sufficient to induce the differentiation of at least some of the pancreatic progenitor cells into endocrine progenitor cells. In some embodiments, S3 media can be used as a suitable culture medium for differentiating pancreatic progenitor cells into endocrine progenitor cells.

In some embodiments, contacting the pancreatic progenitor cells is effected in suspension culture. In some embodiments, the suspension culture is maintained in a spinner flask. In some embodiments, the period of time is at least 5 days. In some embodiments, the suspension culture is replenished every other day. In some embodiments, the at least one BMP signaling pathway inhibitor is added to the suspension culture on the first day of Stage 4 only (e.g., on day 8 of the differentiation protocol).

In some embodiments, endocrine progenitor cells can be obtained by differentiating at least some of the pancreatic progenitor cells in a population into endocrine progenitor cells, e.g., by contacting the pancreatic progenitor cells with LDN193189, to induce the differentiation of at least some of the pancreatic progenitor cells into endocrine progenitor cells, wherein the endocrine progenitor cells express at least one marker characteristic of endocrine progenitor cells.

Polyhormonal cells of use herein can be derived from any source or generated in accordance with any suitable protocol. In some aspects endocrine progenitor cells are differentiated to polyhormonal cells. In some aspects the polyhormonal cells are further differentiated, e.g., by induction or maturation to SC-α cells.

In some aspects, a method of producing a polyhormonal cell from an endocrine progenitor cell comprises contacting a population of cells (e.g., under conditions that promote cell clustering) comprising endocrine progenitor cells with at least one TGF-β signaling pathway inhibitor to induce the differentiation of at least some of the endocrine progenitor cells into polyhormonal cells.

The disclosure contemplates the use of any TGF-β signaling pathway inhibitor that induces endocrine progenitor cells to differentiate into polyhormonal cells. In some embodiments, the TGF-β signaling pathway inhibitor comprises Alk5 inhibitor II. Other examples of TGF-β signaling pathway inhibitors include analogs or derivatives of ALK5 inhibitor II (e.g., compounds of Formula I as described in U.S. Patent Publication No. 2012/0021519, incorporated herein by reference), A 83-01, SB 431542, D 4476, GW 788388, LY 364947, LY 580276, SB 525334, SB 505124, SD 208, GW 6604, and GW 788388.

In some embodiments, the endocrine progenitor cells are contacted with the at least one TGF-β signaling pathway inhibitor at a concentration of between 0.1 uM-50 uM. In some embodiments, the endocrine progenitor cells are contacted with the at least one TGF-β signaling pathway inhibitor at a concentration of 10 uM. In some embodiments, the endocrine progenitor cells are contacted with the at least one TGF-β signaling pathway inhibitor at a concentration of 2 uM, 3 uM, 4 uM, 5 uM, 6 uM, 7 uM, 8 uM, or 9 uM. In some embodiments, the endocrine progenitor cells are contacted with the at least one TGF-β signaling pathway inhibitor at a concentration of 9.1 uM, 9.2 uM, 9.3 uM, 9.4 uM, 9.5 uM, 9.6 uM, 9.7 uM, 9.8 uM or 9.9 uM. In some embodiments, the endocrine progenitor cells are contacted with the at least one TGF-β signaling pathway inhibitor at a concentration of 11 uM, 12 uM, 13 uM, 14 uM, 15 uM, 16 uM, 17 uM, 18 uM, or 19 uM. In some embodiments, the endocrine progenitor cells are contacted with the at least one TGF-β signaling pathway inhibitor at a concentration of 10.1 uM, 10.2 uM, 10.3 uM, 10.4 uM, 10.5 uM, 10.6 uM, 10.7 uM, 10.8 uM or 10.9 uM.

Generally, the endocrine progenitor cells are maintained in a suitable culture medium (e.g., suspension culture) for a period of time sufficient to induce the differentiation of at least some of the pancreatic progenitor cells into polyhormonal cells. In some embodiments, S3 media can be used as a suitable culture medium for differentiating endocrine progenitor cells into polyhormonal cells.

In some embodiments, contacting the endocrine progenitor cells is effected in suspension culture. In some embodiments, the suspension culture is maintained in a spinner flask. In some embodiments, the period of time is at least 7 days. In some embodiments, the suspension culture is replenished every other day. In some embodiments, the at least one TGF-β signaling pathway inhibitor is added to the suspension culture.

In some embodiments, polyhormonal cells can be obtained by differentiating at least some of the endocrine progenitor cells in a population into polyhormonal cells, e.g., by contacting the endocrine progenitor cells with Alk5i, to induce the differentiation of at least some of the endocrine progenitor cells into polyhormonal cells.

In some embodiments the polyhormonal cells comprise cells that express pro/insulin and pro/glucagon. In some aspects the polyhormonal cells express pro/insulin, but do not secrete insulin. In some aspects the polyhormonal cells secrete glucagon under low glucose conditions. In some embodiments the polyhormonal cells express one or more markers of alpha cells, and in some embodiments do not express markers of beta cells. In some embodiments polyhormonal cells express one or more of ARX, IRX1, and IRX2. In some embodiments the polyhormonal cells do not express beta cells markers NKX6-1, PDX1, and PAX4.

SC-α cells of use herein can be derived from any source or generated in accordance with any suitable protocol. In some aspects polyhormonal cells are differentiated or matured to SC-α cells.

In some aspects a method of producing a SC-α cell from a polyhormonal cell comprises transplanting a population of cells comprising polyhormonal cells in a subject and maintaining the population of cells in vivo for a period of time that allows the polyhormonal cells to mature into SC-α cells. In some aspects the transplanted cells are maintained for at least 14 days, at least 28 days, or at least 56 days in vivo. In some embodiments the polyhormonal cells mature to monohormonal pro/glucagon expressing cells (e.g., SC-α cells).

In some aspects a method of producing a SC-α cell from a polyhormonal cell comprises culturing a population of cells comprising polyhormonal cells in vitro for a period of time that allows the polyhormonal cells to mature into SC-α cells. In some aspects the polyhormonal cells are cultured in vitro for at least 14 days, at least 21 days, or at least 28 days. In some embodiments the polyhormonal cells mature to monohormonal pro/glucagon expressing cells (e.g., SC-α cells).

In some embodiments a method of producing a SC-α cell from a polyhormonal cell comprises contacting a population of cells (e.g., under conditions that promote cell clustering) comprising polyhormonal cells with at least one protein kinase C (PKC) activator to induce the differentiation or maturation of at least some of the polyhormonal cells into SC-α cells.

The disclosure contemplates the use of any PKC activator that induces polyhormonal cells to differentiate into SC-α cells. In some embodiments, the PKC activator comprises PdbU. In some embodiments, the PKC activator is selected from the group consisting of PdbU, Bryostatin 1, FR236924, Pseudo RACK1, SC-9, TPPB, PEP005, and combinations thereof. Other examples of PKC activators include TPB, cyclopropanated polyunsaturated fatty acids, cyclopropanated monounsaturated fatty acids, cyclopropanated polyunsaturated fatty alcohols, cyclopropanated monounsaturated fatty alcohols, cyclopropanated polyunsaturated fatty acid esters, cyclopropanated monounsaturated fatty acid esters, cyclopropanated polyunsaturated fatty acid sulfates, cyclopropanated monounsaturated fatty acid sulfates, cyclopropanated polyunsaturated fatty acid phosphates, cyclopropanated monounsaturated fatty acid phosphates, macrocyclic lactones, DAG derivatives, isoprenoids, octylindolactam V, gnidimacrin, iripallidal, ingenol, napthalenesulfonamides, diacylglycerol kinase inhibitors, fibroblast growth factor 18 (FGF-18), insulin growth factor, hormones, and growth factor activators, as described in WIPO Publication No. WO/2013/071282, incorporated herein by reference.

In some embodiments, the polyhormonal cells are contacted with PKC activator at a concentration of between 50 nM-5000 nM. In some embodiments, the polyhormonal cells are contacted with the PKC activator at a concentration of 500 nM. In some embodiments, the polyhormonal cells are contacted with the PKC activator at a concentration of 100 nM, 150 nM, 200 nM, 250 nM, 300 nM, 350 nM, 400 nM, 450 nM, 460 nM, 470 nM, 480 nM, or 490 nM. In some embodiments, the polyhormonal cells are contacted with the PKC activator at a concentration of 491 nM, 492 nM, 493 nM, 494 nM, 495 nM, 496 nM, 497 nM, 498 nM, or 499 nM. In some embodiments, the polyhormonal cells are contacted with the PKC activator at a concentration of 600 nM, 700 nM, 800 nM, 900 nM, 1000 nM, 1100 nM, 1200 nM, 1300 nM, 1400 nM, 1500 nM, 1600 nM, 1700 nM, 1800 nM, 1900 nM, or 2000 nM. In some embodiments, the polyhormonal cells are contacted with the PKC activator at a concentration of 501 nM, 502 nM, 503 nM, 504 nM, 505 nM, 506 nM, 507 nM, 508 nM, or 509 nM, 510 nM, 520 nM, 530 nM, 540 nM, 550 nM, 560 nM, 570 nM, 580 nM, or 590 nM.

Generally, the polyhormonal cells are maintained in a suitable culture medium (e.g., suspension culture) for a period of time sufficient to induce the differentiation of at least some of the polyhormonal cells into SC-α cells. In some embodiments, S3 media can be used as a suitable culture medium for differentiating polyhormonal cells into SC-α cells.

In some embodiments, contacting the polyhormonal cells is effected in suspension culture. In some embodiments, the suspension culture is maintained in a spinner flask. In some embodiments, the period of time is at least 28 days. In some embodiments, the suspension culture is replenished every other day. In some embodiments, the at least one PKC activator is added to the suspension culture.

In some embodiments, SC-α cells can be obtained by differentiating at least some of the polyhormonal cells in a population into SC-α cells, e.g., by contacting the polyhormonal cells with PdbU, to induce the differentiation or maturation of at least some of the polyhormonal cells into SC-α cells.

In some aspects of the disclosure SC-α cells are identified within a population of endocrine cells. In some aspects a population of endocrine cells is screened to identify SC-α cells included within the population. In some embodiments the population of endocrine cells are screened using single-cell sequencing (e.g., high throughput single-cell RNA sequencing) to identify the cells located within the population. In some aspects the SC-α cells are identified as expressing at least one of ARX, IRX1, and IRX2. In some aspects SC-α cells co-express ARX, IRX1, and IRX2.

In some aspects of the disclosure, a stem cell-derived alpha cell (SC-α) is provided. The SC-α cells disclosed herein share many distinguishing features of native alpha cells, but are different in certain aspects. In some embodiments, the SC-α cell is non-native. As used herein, “non-native” means that the SC-α cells are markedly different in certain aspects from alpha cells which exist in nature, i.e., native alpha cells. It should be appreciated, however, that these marked differences may result in the SC-α cells exhibiting certain differences, but the SC-α cells may still behave in a similar manner to native alpha cells with certain functions altered (e.g., improved) compared to native alpha cells. In some embodiments SC-α cells are derived from a polyhormonal intermediate, while an adult alpha cell (e.g., native alpha cell) does not. In some embodiments SC-α cells do not express high levels of C10orf10 (DEPP1) which native alpha cells do express high levels of C10orf10 (DEPP1). In some embodiments SC-α cells do not secrete higher levels of glucagon in response to arginine, unlike native alpha cells.

The SC-α cells of the disclosure share many characteristic features of alpha cells which are important for normal alpha cell function. In some embodiments, the SC-α cell is capable of secreting glucagon in response to glucose and/or glucagon secretagogues. Glucagon may be secreted in response to low glucose levels. In some embodiments the SC-α cell does not express c-peptide. An SC-α cell may be monohormonal. In some embodiments the SC-α cell comprises one or more glucagon granules. The SC-α cell may comprise secretory granules having a similar morphology to alpha cells with an average size of 220 nm. In some aspects the SC-α cell exhibits an ultastructure similar to cadaveric alpha cells. In some embodiments the SC-α cell expresses one or more of the following genes: ARX, IRX1, IRX2, DPP4, GCG, PSCK2, Pou6F2, FEV, TTR, and GC. In some embodiments the SC-α cell co-expresses ARX, IRX1, and IRX2.

The SC-α cells are differentiated in vitro from any starting cell as the invention is not intended to be limited by the starting cell from which the SC-α cells are derived. Exemplary starting cells include, without limitation, polyhormonal cells or any precursor thereof such as an endocrine cell, pancreatic progenitor cell, gut tube endoderm cell, definitive endoderm cell, a pluripotent stem cell, an embryonic stem cell, and induced pluripotent stem cell. In some embodiments, the SC-α cells are differentiated in vitro from a reprogrammed cell, a partially reprogrammed cell (i.e., a somatic cell, e.g., a fibroblast which has been partially reprogrammed such that it exists in an intermediate state between an induced pluripotency cell and the somatic cell from which it has been derived), a transdifferentiated cell. In some embodiments, the SC-α cells disclosed herein can be differentiated in vitro from a polyhormonal cell or a precursor thereof. In some embodiments, the SC-α cell is differentiated in vitro from a precursor selected from the group consisting of an endocrine progenitor cell, a pancreatic progenitor cell, a definitive endoderm cell, and a pluripotent stem cell. In some embodiments, the pluripotent stem cell is selected from the group consisting of an embryonic stem cell and induced pluripotent stem cell. In some embodiments, the SC-α cell or the pluripotent stem cell from which the SC-α cell is derived is human. In some embodiments, the SC-α cell is human.

In some embodiments, the SC-α cell is not genetically modified. In some embodiments, the SC-α cell obtains the features it shares in common with native alpha cells in the absence of a genetic modification of cells. In some embodiments, the SC-α cell is genetically modified.

In some aspects, the disclosure provides a cell line comprising a SC-α cell described herein. In some aspects, the disclosure provides an artificial islet or pancrease (e.g., an SC-islet) comprising a SC-α cells described herein. In some embodiments the SC-islet further comprises SC-β cells and/or SC-δ cells. The SC-β cells and/or SC-δ cells may be obtained by any methods known by those of skill in the art. For example, the stem cell-derived cells may be obtained using the differentiation protocols described in WO 2015/002724 and WO 2014/201167, both of which are incorporated herein by reference in their entirety.

An artificial pancreas is a device that encapsulates and nurtures islets of Langerhans to replace the islets and α, β and/or δ cells destroyed by type 1 diabetes. An artificial pancreas may contain a million islets or more, and may be implanted in the peritoneal cavity or under the skin where it can respond to changing blood glucose levels by releasing hormones, such as glucagon, somatostatin, and/or insulin. An artificial pancreas may be made using living (e.g., glucose-sensing and glucagon secreting islets) and non-living components (e.g., to shield the islets from the diabetic subject's body and its destructive immune mechanism while permitting the islets to thrive).

The present invention contemplates using SC-α cells, alone or in combination with SC-β cells and/or SC-δ cells, in any artificial pancreas. In some aspects, the artificial pancreas comprises microencapsulated or coated islets comprising SC-α cells generated according to the methods described herein. In some aspects, the artificial pancreas comprises a macroencapsulation device into which islet cells comprising SC-α cells generated according to the methods herein are grouped together and encapsulated. In some aspects, the macroencapsulation device comprises a PVA hydrogel sheet for an artificial pancreas of the present invention. In some aspects, the artificial islet comprises SC-α cells generated according to the methods herein, along with other islet cells (β, δ, etc.) in the form of an islet sheet. The islet sheet comprises a layer of artificial human islets comprising the SC-α cells macroencapsulated within a membrane (e.g., of ultra-pure alginate). The sheet membrane is reinforced with mesh and may be coated on the surface to prevent or minimize contact between the cells encapsulated inside and the transplantation recipient's host immune response. Oxygen, glucose, and other nutrients readily diffuse into the sheet through the membrane nurturing the islets, and hormones, such as insulin, readily diffuse out. Additional examples of membranes designed for macroencapsulation/implantation of an artificial islet or pancreas are known to those of skill in the art.

In some embodiments, the cells described herein, e.g. a population of SC-α cells are transplantable, e.g., a population of SC-α cells can be administered to a subject. In some embodiments, the subject who is administered a population of SC-α cells is the same subject from whom a pluripotent stem cell used to differentiate into a SC-α cell was obtained (e.g. for autologous cell therapy). In some embodiments, the subject is a different subject In some embodiments, a subject is suffering from diabetes such as type I diabetes, or is a normal subject. For example, the cells for transplantation (e.g. a composition comprising a population of SC-α cells) can be a form suitable for transplantation, e.g., organ transplantation.

The method can further include administering the cells to a subject in need thereof, e.g., a mammalian subject, e.g., a human subject. The source of the cells can be a mammal, preferably a human. The source or recipient of the cells can also be a non-human subject, e.g., an animal model. The term “mammal” includes organisms, which include mice, rats, cows, sheep, pigs, rabbits, goats, horses, monkeys, dogs, cats, and preferably humans. Likewise, transplantable cells can be obtained from any of these organisms, including a non-human transgenic organism. In one embodiment, the transplantable cells are genetically engineered, e.g., the cells include an exogenous gene or have been genetically engineered to inactivate or alter an endogenous gene.

A composition comprising a population of SC-α cells can be administered to a subject using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

For administration to a subject, a cell population produced by the methods as disclosed herein, e.g. a population of SC-α cells can be administered to a subject, for example in pharmaceutically acceptable compositions. These pharmaceutically acceptable compositions comprise a therapeutically-effective amount a population of SC-α cells as described above, formulated together with one or more pharmaceutically acceptable carriers (additives) and/or diluents.

As described in detail below, the pharmaceutical compositions of the present invention can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

The phrase “therapeutically-effective amount” as used herein in respect to a population of cells means that amount of relevant cells in a population of cells, e.g., SC-α cells, or composition comprising SC-α cells of the present invention which is effective for producing some desired therapeutic effect in at least a sub-population of cells in an animal at a reasonable benefit/risk ratio applicable to any medical treatment. For example, an amount of a population of SC-α cells administered to a subject that is sufficient to produce a statistically significant, measurable change in at least one symptom of Type 1, Type 1.5 or Type 2 diabetes, such as glycosylated hemoglobin level, fasting blood glucose level, hypoinsulinemia, etc. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

As used herein, the term “administer” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered by intravenous infusion or injection.

By “treatment”, “prevention” or “amelioration” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, the symptoms of a disease or disorder are alleviated by at least 5%, at least 10%, at least 20%, at least 30%, at least 40%, or at least 50%.

Treatment of diabetes is determined by standard medical methods. A goal of diabetes treatment is to bring sugar levels down to as close to normal as is safely possible. Commonly set goals are 80-120 milligrams per deciliter (mg/dl) before meals and 100-140 mg/dl at bedtime. A particular physician may set different targets for the patent, depending on other factors, such as how often the patient has low blood sugar reactions. Useful medical tests include tests on the patient's blood and urine to determine blood sugar level, tests for glycosylated hemoglobin level (HbA1c; a measure of average blood glucose levels over the past 2-3 months, normal range being 4-6%), tests for cholesterol and fat levels, and tests for urine protein level. Such tests are standard tests known to those of skill in the art (see, for example, American Diabetes Association, 1998). A successful treatment program can also be determined by having fewer patients in the program with complications relating to diabetes, such as diseases of the eye, kidney disease, or nerve disease.

Delaying the onset of diabetes in a subject refers to delay of onset of at least one symptom of diabetes, e.g., hyperglycemia, hypoinsulinemia, diabetic retinopathy, diabetic nephropathy, blindness, memory loss, renal failure, cardiovascular disease (including coronary artery disease, peripheral artery disease, cerebrovascular disease, atherosclerosis, and hypertension), neuropathy, autonomic dysfunction, hyperglycemic hyperosmolar coma, or combinations thereof, for at least 1 week, at least 2 weeks, at least 1 month, at least 2 months, at least 6 months, at least 1 year, at least 2 years, at least 5 years, at least 10 years, at least 20 years, at least 30 years, at least 40 years or more, and can include the entire lifespan of the subject.

In certain embodiments, the subject is a mammal, e.g., a primate, e.g., a human. The terms, “patient” and “subject” are used interchangeably herein. Preferably, the subject is a mammal. The mammal can be a human, non-human primate, mouse, rat, dog, cat, horse, or cow, but are not limited to these examples. Mammals other than humans can be advantageously used as subjects that represent animal models of Type 1 diabetes, Type 2 Diabetes Mellitus, or pre-diabetic conditions. In addition, the methods described herein can be used to treat domesticated animals and/or pets. A subject can be male or female. A subject can be one who has been previously diagnosed with or identified as suffering from or having diabetes (e.g., Type 1 or Type 2), one or more complications related to diabetes, or a pre-diabetic condition, and optionally, but need not have already undergone treatment for diabetes, the one or more complications related to diabetes, or the pre-diabetic condition. A subject can also be one who is not suffering from diabetes or a pre-diabetic condition. A subject can also be one who has been diagnosed with or identified as suffering from diabetes, one or more complications related to diabetes, or a pre-diabetic condition, but who show improvements in known diabetes risk factors as a result of receiving one or more treatments for diabetes, one or more complications related to diabetes, or the pre-diabetic condition. Alternatively, a subject can also be one who has not been previously diagnosed as having diabetes, one or more complications related to diabetes, or a pre-diabetic condition. For example, a subject can be one who exhibits one or more risk factors for diabetes, complications related to diabetes, or a pre-diabetic condition, or a subject who does not exhibit diabetes risk factors, or a subject who is asymptomatic for diabetes, one or more diabetes-related complications, or a pre-diabetic condition. A subject can also be one who is suffering from or at risk of developing diabetes or a pre-diabetic condition. A subject can also be one who has been diagnosed with or identified as having one or more complications related to diabetes or a pre-diabetic condition as defined herein, or alternatively, a subject can be one who has not been previously diagnosed with or identified as having one or more complications related to diabetes or a pre-diabetic condition.

As used herein, the phrase “subject in need of SC-α cells” refers to a subject who is diagnosed with or identified as suffering from, having or at risk for developing diabetes (e.g., Type 1, Type 1.5 or Type 2), one or more complications related to diabetes, or a pre-diabetic condition.

A subject in need of a population of SC-α cells can be identified using any method used for diagnosis of diabetes. For example, Type 1 diabetes can be diagnosed using a glycosylated hemoglobin (AlC) test, a random blood glucose test and/or a fasting blood glucose test. Parameters for diagnosis of diabetes are known in the art and available to skilled artisan without much effort.

In some embodiments, the methods of the invention further comprise selecting a subject identified as being in need of additional SC-α cells. A subject in need a population of SC-α cells can be selected based on the symptoms presented, such as symptoms of type 1, type 1.5 or type 2 diabetes. Exemplary symptoms of diabetes include, but are not limited to, excessive thirst (polydipsia), frequent urination (polyuria), extreme hunger (polyphagia), extreme fatigue, weight loss, hyperglycemia, low levels of insulin, high blood sugar (e.g., sugar levels over 250 mg, over 300 mg), presence of ketones present in urine, fatigue, dry and/or itchy skin, blurred vision, slow healing cuts or sores, more infections than usual, numbness and tingling in feet, diabetic retinopathy, diabetic nephropathy, blindness, memory loss, renal failure, cardiovascular disease (including coronary artery disease, peripheral artery disease, cerebrovascular disease, atherosclerosis, and hypertension), neuropathy, autonomic dysfunction, hyperglycemic hyperosmolar coma, and combinations thereof.

In some embodiments, a composition comprising a population of SC-α cells for administration to a subject can further comprise a pharmaceutically active agent, such as those agents known in the art for treatment of diabetes and or for having anti-hyperglycemic activities, for example, inhibitors of dipeptidyl peptidase 4 (DPP-4) (e.g., Alogliptin, Linagliptin, Saxagliptin, Sitagliptin, Vildagliptin, and Berberine), biguanides (e.g., Metformin, Buformin and Phenformin), peroxisome proliferator-activated receptor (PPAR) modulators such as thiazolidinediones (TZDs) (e.g., Pioglitazone, Rivoglitazone, Rosiglitazone and Troglitazone), dual PPAR agonists (e.g., Aleglitazar, Muraglitazar and Tesaglitazar), sulfonylureas (e.g., Acetohexamide, Carbutamide, Chlorpropamide, Gliclazide, Tolbutamide, Tolazamide, Glibenclamide (Glyburide), Glipizide, Gliquidone, Glyclopyramide, and Glimepiride), meglitinides (“glinides”) (e.g., Nateglinide, Repaglinide and Mitiglinide), glucagon-like peptide-1 (GLP-1) and analogs (e.g., Exendin-4, Exenatide, Liraglutide, Albiglutide), insulin and insulin analogs (e.g., Insulin lispro, Insulin aspart, Insluin glulisine, Insulin glargine, Insulin detemir, Exubera and NPH insulin), alpha-glucosidase inhibitors (e.g., Acarbose, Miglitol and Voglibose), amylin analogs (e.g. Pramlintide), Sodium-dependent glucose cotransporter T2 (SGLT T2) inhibitors (e.g., Dapgliflozin, Remogliflozin and Sergliflozin) and others (e.g. Benfluorex and Tolrestat).

In type 1 diabetes, a cells are undesirably destroyed by continued autoimmune response. Thus, this autoimmune response can be attenuated by use of compounds that inhibit or block such an autoimmune response. In some embodiments, a composition comprising a population of SC-α cells for administration to a subject can further comprise a pharmaceutically active agent which is a immune response modulator. As used herein, the term “immune response modulator” refers to a compound (e.g., a small-molecule, antibody, peptide, nucleic acid, or gene therapy reagent) that inhibits autoimmune response in a subject. Without wishing to be bound by theory, an immune response modulator inhibits the autoimmune response by inhibiting the activity, activation, or expression of inflammatory cytokines (e.g., IL-12, IL-23 or IL-27), or STAT-4. Exemplary immune response modulators include, but are not limited to, members of the group consisting of Lisofylline (LSF) and the LSF analogs and derivatives described in U.S. Pat. No. 6,774,130, contents of which are herein incorporated by reference in their entirety.

A composition comprising SC-α cells can be administrated to the subject in the same time, of different times as the administration of a pharmaceutically active agent or composition comprising the same. When administrated at different times, the compositions comprising a population of SC-α cells and/or pharmaceutically active agent for administration to a subject can be administered within 5 minutes, 10 minutes, 20 minutes, 60 minutes, 2 hours, 3 hours, 4, hours, 8 hours, 12 hours, 24 hours of administration of the other. When a composition comprising a population of SC-α cells and a composition comprising a pharmaceutically active agent are administered in different pharmaceutical compositions, routes of administration can be different. In some embodiments, a subject is administered a composition comprising SC-α cells. In other embodiments, a subject is administered a composition comprising a pharmaceutically active agent. In another embodiment, a subject is administered a compositions comprising a population of SC-α cells mixed with a pharmaceutically active agent. In another embodiment, a subject is administered a composition comprising a population of SC-α cells and a composition comprising a pharmaceutically active agent, where administration is substantially at the same time, or subsequent to each other.

Toxicity and therapeutic efficacy of administration of a compositions comprising a population of SC-α cells can be determined by standard pharmaceutical procedures in cell cultures or experimental animals, e.g., for determining the LD50 (the dose lethal to 50% of the population) and the ED50 (the dose therapeutically effective in 50% of the population). Compositions comprising a population of SC-α cells that exhibit large therapeutic indices are preferred.

The amount of a composition comprising a population of SC-α cells can be tested using several well-established animal models.

The non-obese diabetic (NOD) mouse carries a genetic defect that results in insulitis showing at several weeks of age (Yoshida et al., Rev. Immunogenet. 2:140, 2000). 60-90% of the females develop overt diabetes by 20-30 weeks. The immune-related pathology appears to be similar to that in human Type I diabetes. Other models of Type I diabetes are mice with transgene and knockout mutations (Wong et al., Immunol. Rev. 169:93, 1999). A rat model for spontaneous Type I diabetes was recently reported by Lenzen et al. (Diabetologia 44:1189, 2001). Hyperglycemia can also be induced in mice (>500 mg glucose/dL) by way of a single intraperitoneal injection of streptozotocin (Soria et al., Diabetes 49:157, 2000), or by sequential low doses of streptozotocin (Ito et al., Environ. Toxicol. Pharmacol. 9:71, 2001). To test the efficacy of implanted islet cells, the mice are monitored for return of glucose to normal levels (<200 mg/dL).

Larger animals provide a good model for following the sequelae of chronic hyperglycemia. Dogs can be rendered insulin-dependent by removing the pancreas (J. Endocrinol. 158:49, 2001), or by feeding galactose (Kador et al., Arch. Opthalmol. 113:352, 1995). There is also an inherited model for Type I diabetes in keeshond dogs (Am. J. Pathol. 105:194, 1981). Early work with a dog model (Banting et al., Can. Med. Assoc. J. 22:141, 1922) resulted in a couple of Canadians making a long ocean journey to Stockholm in February of 1925.

In some embodiments, data obtained from the cell culture assays and in animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

The therapeutically effective dose of a composition comprising a population of SC-α cells can also be estimated initially from cell culture assays. A dose may be formulated in animal models in vivo to achieve a secretion of glucagon at a concentration which is appropriate in response to circulating glucose in the plasma. Alternatively, the effects of any particular dosage can be monitored by a suitable bioassay.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the SC-α cells. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

In another aspect of the invention, the methods provide use of an isolated population of SC-α cells as disclosed herein. In one embodiment of the invention, an isolated population of SC-α cells as disclosed herein may be used for the production of a pharmaceutical composition, for the use in transplantation into subjects in need of treatment, e.g. a subject that has, or is at risk of developing diabetes, for example but not limited to subjects with congenital and acquired diabetes. In one embodiment, an isolated population of SC-α cells may be genetically modified. In another aspect, the subject may have or be at risk of diabetes and/or metabolic disorder. In some embodiments, an isolated population of SC-α cells as disclosed herein may be autologous and/or allogeneic. In some embodiments, the subject is a mammal, and in other embodiments the mammal is a human.

The use of an isolated population of SC-α cells as disclosed herein provides advantages over existing methods because the population of SC-α cells can be differentiated from endocrine progenitor cells or precursors thereof derived from stem cells, e.g. iPS cells obtained or harvested from the subject administered an isolated population of SC-α cells. This is highly advantageous as it provides a renewable source of SC-α cells with can be differentiated from stem cells to endocrine progenitor cells by methods commonly known by one of ordinary skill in the art, and then further differentiated by the methods described herein to pancreatic α-like cells or cells with pancreatic α cell characteristics, for transplantation into a subject, in particular a substantially pure population of mature pancreatic α-like cells that do not have the risks and limitations of cells derived from other systems.

In another embodiment, an isolated population of SC-α cells can be used as models for studying properties for differentiation into glucagon-producing cells, e.g. to pancreatic α cells or pancreatic α-like cells, or pathways of development of cells of endoderm origin into pancreatic α cells.

In some embodiments, the endocrine progenitor cells or SC-α cells may be genetically engineered to comprise markers operatively linked to promoters that are expressed when a marker is expressed or secreted, for example, a marker can be operatively linked to an glucagon promoter, so that the marker is expressed when the endocrine progenitor cells or precursors thereof differentiate into SC-α cells which express and secrete glucagon. In some embodiments, a population of SC-α cells can be used as a model for studying the differentiation pathway of cells which differentiate into islet cells or pancreatic α-like cells.

In other embodiments, the glucagon-producing, glucose responsive cells can be used as models for studying the role of islet cells in the pancreas and in the development of diabetes and metabolic disorders. In some embodiments, the SC-α cells can be from a normal subject, or from a subject which carries a mutation and/or polymorphism. In some embodiments, the SC-α cells may be genetically engineered to correct the polymorphism prior to being administered to a subject in the therapeutic treatment of a subject with diabetes. In some embodiments, the SC-α cells may be genetically engineered to carry a mutation and/or polymorphism.

One embodiment of the invention relates to a method of treating diabetes or a metabolic disorder in a subject comprising administering an effective amount of a composition comprising a population of SC-α cells as disclosed herein to a subject with diabetes and/or a metabolic disorder. In a further embodiment, the invention provides a method for treating diabetes, comprising administering a composition comprising a population of SC-α cells as disclosed herein to a subject that has, or has an increased risk of developing diabetes in an effective amount sufficient to produce glucagon in response to increased blood glucose levels.

In one embodiment of the above methods, the subject is a human and a population of SC-α cells as disclosed herein are human cells. In some embodiments, the invention contemplates that a population of SC-α cells as disclosed herein are administered directly to the pancreas of a subject, or is administered systemically. In some embodiments, a population of SC-α cells as disclosed herein can be administered to any suitable location in the subject, for example in a capsule in the blood vessel or the liver or any suitable site where administered the population of SC-α cells can secrete glucagon in response to increased glucose levels in the subject.

The present invention is also directed to a method of treating a subject with diabetes or a metabolic disorder which occurs as a consequence of genetic defect, physical injury, environmental insult or conditioning, bad health, obesity and other diabetes risk factors commonly known by a person of ordinary skill in the art. Efficacy of treatment of a subject administered a composition comprising a population of SC-α cells can be monitored by clinically accepted criteria and tests, which include for example, (i) Glycated hemoglobin (A1C) test, which indicates a subjects average blood sugar level for the past two to three months, by measuring the percentage of blood sugar attached to hemoglobin, the oxygen-carrying protein in red blood cells. The higher your blood sugar levels, the more hemoglobin has sugar attached. An A1C level of 6.5 percent or higher on two separate tests indicates the subject has diabetes. A test value of 6-6.5% suggest the subject has prediabetes. (ii) Random blood sugar test. A blood sample will be taken from the subject at a random time, and a random blood sugar level of 200 milligrams per deciliter (mg/dL)-11.1 millimoles per liter (mmol/L), or higher indicated the subject has diabetes. (iii) Fasting blood sugar test. A blood sample is taken from the subject after an overnight fast. A fasting blood sugar level between 70 and 99 mg/dL (3.9 and 5.5 mmol/L) is normal. If the subjects fasting blood sugar levels is 126 mg/dL (7 mmol/L) or higher on two separate tests, the subject has diabetes. A blood sugar level from 100 to 125 mg/dL (5.6 to 6.9 mmol/L) indicates the subject has prediabetes. (iv) Oral glucose tolerance test. A blood sample will be taken after the subject has fasted for at least eight hours or overnight and then ingested a sugary solution, and the blood sugar level will be measured two hours later. A blood sugar level less than 140 mg/dL (7.8 mmol/L) is normal. A blood sugar level from 140 to 199 mg/dL (7.8 to 11 mmol/L) is considered prediabetes. This is sometimes referred to as impaired glucose tolerance (IGT). A blood sugar level of 200 mg/dL (11.1 mmol/L) or higher may indicate diabetes.

In some embodiments, the effects of administration of a population of SC-α cells as disclosed herein to a subject in need thereof is associated with improved exercise tolerance or other quality of life measures, and decreased mortality. The effects of cellular therapy with a population of SC-α cells can be evident over the course of days to weeks after the procedure. However, beneficial effects may be observed as early as several hours after the procedure, and may persist for several years. In some embodiments, the effects of cellular therapy with a population of SC-α cells occurs within two weeks after the procedure.

In some embodiments, a population of SC-α cells as disclosed herein may be used for tissue reconstitution or regeneration in a human patient or other subject in need of such treatment. In some embodiments compositions of populations of SC-α cells can be administered in a manner that permits them to graft or migrate to the intended tissue site and reconstitute or regenerate the functionally deficient area. Special devices are available that are adapted for administering cells capable of reconstituting a population of α cells in the pancreas or at an alternative desired location. Accordingly, the SC-α cells may be administered to a recipient subject's pancreas by injection, or administered by intramuscular injection.

In some embodiments, compositions comprising a population of SC-α cells as disclosed herein have a variety of uses in clinical therapy, research, development, and commercial purposes. For therapeutic purposes, for example, a population of SC-α cells as disclosed herein may be administered to enhance glucagon production in response to increases in blood glucose level for any perceived need, such as an inborn error in metabolic function, the effect of a disease condition (e.g. diabetes), or the result of significant trauma (i.e. damage to the pancreas or loss or damage to islet cells). In some embodiments, a population of SC-α cells as disclosed herein are administered to the subject not only to help restore function to damaged or otherwise unhealthy tissues, but also to facilitate remodeling of the damaged tissues.

In some aspects of the disclosure, single-cell sequencing (e.g., high throughput single-cell RNA sequencing) is used to provide a detailed characterization of the full transcriptomes of all cell populations produced using an in vitro alpha cell differentiation protocol. In some embodiments, specific genes are identified as enriching a single population of cells or combination of cells. For example, ARX, IRK1, and IRK2 are transcription factors known to be essential for SC-α cell development. By targeting one or more of these factors (e.g., modulating expression of one of the essential factors) using any gene editing tool known to those of skill in the art (e.g., TALENS, CRISPR, etc.) SC-α cell development can be modulated. In some aspects a known essential factor may be inhibited or knocked-out thereby inhibiting SC-α cell development. In other aspects a known essential factor may be activated thereby increasing SC-α cell development. In some embodiments tumor suppressors (e.g., RB1 and/or NF2) may be knocked out or inhibited thereby resulting in uncontrolled growth of stem cells and/or progenitors, and the failure of cells to differentiate.

In some embodiments, one or more transcription factors may be identified as controlling cell fate during a differentiation protocol. For example, PAX4 and ARX are example regulators of SC-β cell and SC-α cell differentiation. The regulators control the fate of the differentiated cells. In some aspects a regulator may be targeted using gene editing (e.g., CRISPR) to modulate expression of the regulator and thereby control the fate of the differentiation process. In some aspects a first regulator may be knocked out or inhibited, while a second regulator may be activated. In some aspects ARX is inhibited or knocked-out and/or PAX4 is activated thereby causing a population of differentiating cells to form SC-β cells. In other aspects ARX is activated and/or PAX4 is knocked out or inhibited thereby causing a population of differentiating cells to form SC-α cells.

### EXAMPLES

### Example 1: Generation of Stem Cell Derived Alpha Cells

The generation of pancreatic cell types from renewable cell sources holds great promise for the development of cell replacement therapies for diabetes. Although much effort has focused on generating pancreatic beta cells, mounting evidence suggests that the glucagon secreting alpha cells are critical to disease progression and treatment. Here the efforts to generate pancreatic alpha cells from stem cells are described. These SC-alpha cells are generated through a polyhormonal intermediate. The generation of these polyhormonal cells is optimized and their expression and secretion profile is characterized. Polyhormonal cells exhibit a transcriptional profile similar to alpha cells and although they produce proinsulin transcript and protein, they do not secrete processed insulin. Upon transplantation, or extended culture in vitro, these polyhormonal cells reduce their proinsulin expression while continuing to express glucagon. Compound screening identified a PKC activator that promotes the maturation process of polyhormonal cells to SC-alpha cells. The resulting SC-alpha cells share an ultrastructure similar to cadaveric alpha cells, express and secrete glucagon in response to glucose and glucagon secretagogues, elevate blood glucose upon transplantation in mice, and protect mice from insulin induced hypoglycemia. These results show that SC-alpha cells faithfully recapitulate central aspects of human alpha cells and may be used as part of islet organoids for cell replacement therapy.

### Generation of Polyhormonal Cells During Directed Differentiation

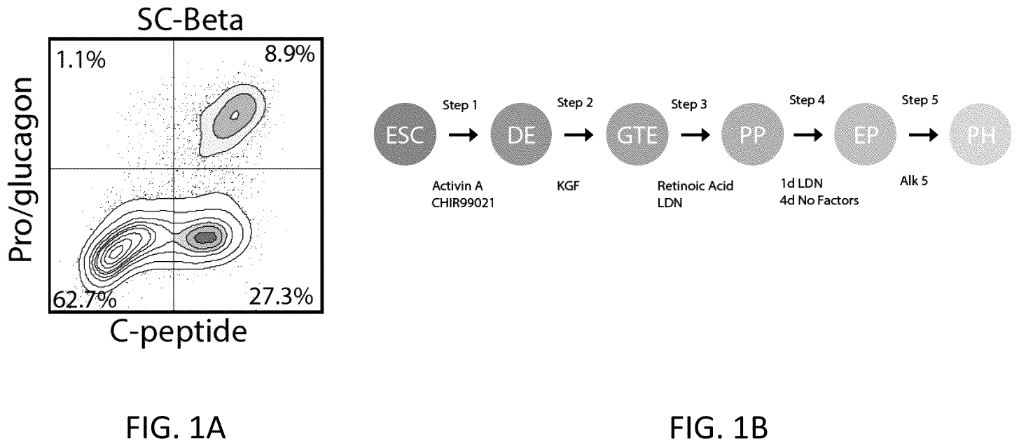

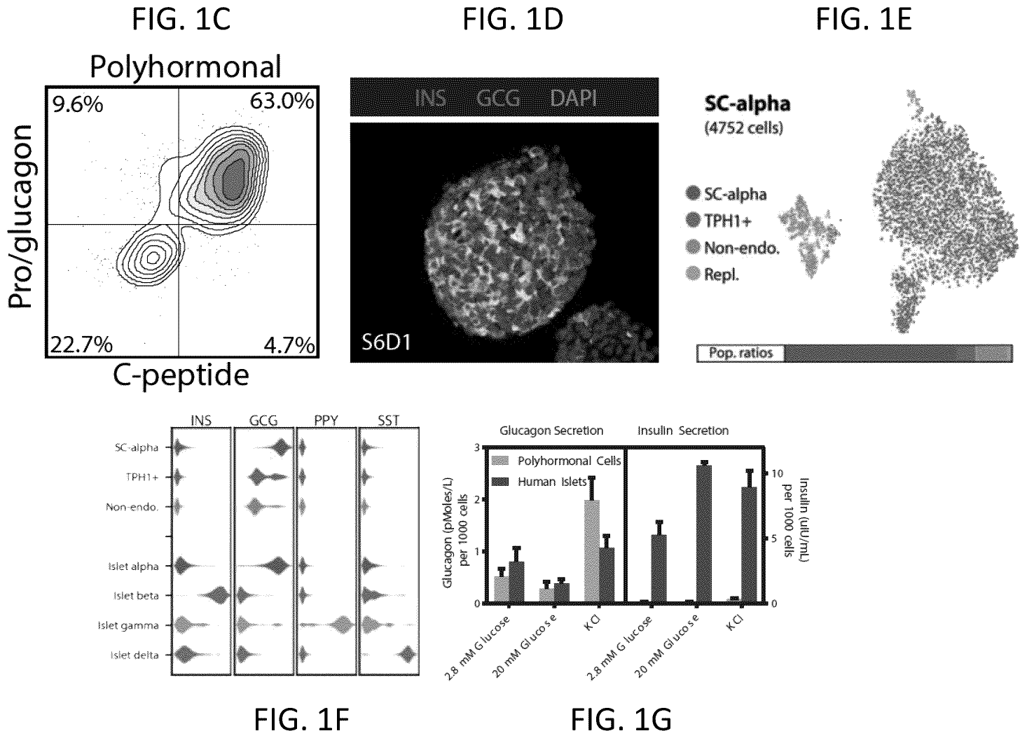

The generation of functional pancreatic beta cells from human pluripotent stem cells using a six-step directed differentiation protocol has previously been reported⁽¹⁰⁾. This protocol generates a population of cells that contains several subpopulations of cells including cell that express markers of pancreatic beta cells as well as other side populations. FIG. 1A shows a typical distribution wherein 27% of the cells are monohormonal glucose responsive beta cells (SC-beta cells) and a second population (9% of the cells) are polyhormonal cells that express both c-peptide and glucagon (FIG. 1A). The presence of polyhormonal cells in pancreatic differentiations has been observed in several previously reported protocols⁽¹⁵⁻¹⁷⁾, however, much remains unknown regarding these polyhormonal cells. Several reports have described polyhormonal cells that exhibit features of immature beta cells⁽¹⁶⁻¹⁹⁾. Beyond the expression of insulin, there has been little evidence to support the similarity of these cells to beta cells or the ability of polyhormonal cells to further differentiate into beta cells. Others have noted that polyhormonal cells are present during development, contribute to alpha cells later in development, express some markers of alpha cells and when transplanted give rise to glucagon expressing cells. Taken together these results suggest that polyhormonal cells can further differentiate into alpha cells⁽⁹⁾. Although we initially became interested in the nature of these polyhormonal cells as a way to improve beta cell differentiations, we now seek to harness their potential in generating glucagon expressing alpha cells.

### Optimization of S1-4 to Generate Polyhormonal Cells

In order to study polyhormonal cells in more detail, we sought to develop a protocol for the efficient production of polyhormonal cells in vitro as marked by co-expression of insulin and glucagon. Because the antibodies used for assessment of these cells recognize epitopes from both proinsulin and processed insulin and proglucagon and processed glucagon respectively, we call these cells pro/insulin+ and pro/glucagon+. Cells that express a combination of pro/insulin and pro/glucagon are considered to be polyhormonal. Previous findings have suggested that the generation of polyhormonal cells results from pancreatic progenitor cells that fail to express Nkx6.1⁽¹⁵⁾. To this end we sought to modify our beta cell protocol to prevent the induction of Nkx6.1 at stage 4 using the HUES8 embryonic stem cell line. Using our previously published protocols as a starting point, we observed that removal of KGF, SANT-1 and treatment of LDN only on day 2 of step 3 of the protocol resulted in a decrease in the percentage of cells expressing Nkx6.1. In addition, we observed that treatment with LDN on day 1 of step 4 and incubation with no factors for the remainder of step 4 resulted in a significant population of chromogranin+/Nkx6.1-cells. Hypothesizing that these changes would have a significant improvement in the induction of polyhormonal cells, we incorporated these modifications into our polyhormonal protocol (FIG. 1). This protocol resulted in a large fraction of the cells co-expressing insulin and glucagon (FIG. 1C). This polyhormonal optimized protocol produces an average of 69±9% polyhormonal cells but continues to express a small percentage (<9%) of monohormonal SC-alpha cells. Immunofluorescent staining confirms a high proportion of cells that express both pro/insulin and pro/glucagon (FIG. 1D) distributed throughout the cell clusters.

Because previous published protocols have shown variability when used on different cell lines⁽²⁰⁾, the robustness of this polyhormonal protocol was evaluated in the 1016 iPS cell line. When differentiated using this cell line, we were able to generate similar percentages of polyhormonal cells. These results demonstrate the ability to direct stem cells to a polyhormonal cells fate using this protocol.

### Polyhormonal Cells Transcriptional Profile

To better characterize polyhormonal cells we investigated their transcriptional signature by single cell RNAseq. Using a microfluidic approach to single cell sequencing named inDrops⁽¹¹⁾, we profiled 2345 cells from our polyhormonal differentiation. Computational analysis of the expression profile from these cells reveals three distinct cell populations (FIG. 1E). As expected from our immunostaining and flow cytometry analysis, we identified a population of cell that express both insulin and glucagon, although the expression of insulin was 1000-fold lower than the expression of glucagon. This polyhormonal population (FIG. 1E, red) expresses a transcriptional signature that is more similar to an alpha cell than to a beta cell. In addition to expressing pro/insulin and pro/glucagon, the polyhormonal cells also express several markers of alpha cells and are missing several key markers for beta cells. For example, polyhormonal cells express ARX, IRX1, and IRX2, transcription factors that are expressed in alpha cells⁽²¹⁾. In contrast, these cells do not express the key beta cell markers NKX6-1, PDX1, and PAX4. FIG. 1F shows the relative expression levels of pancreatic hormones in the polyhormonal population in comparison to the major endocrine cell types from human islets.

In addition to the major polyhormonal population identified from this analysis, two minor cell populations are present including a TPH1 expressing population and a non-endocrine population. The presence of these minor populations and the large number of polyhormonal cells confirms at the transcriptional level our finding that the majority of cells produced with this protocol are polyhormonal cells. Since the transcriptional profile of polyhormonal cells more closely resembles the transcripts found in alpha cells it was hypothesized that this polyhormonal side population may be useful in generating stem cell derived alpha (SC-alpha) cells.

### Polyhormonal Cells Secrete Glucagon but do not Secrete Insulin

Polyhormonal clusters generated with this protocol secrete glucagon under low glucose (2.8 mM) conditions and suppress glucagon secretion in the presence of hyperglycemia (20 mM) as shown in FIG. 1G. Although these cells express pro/insulin, the c-peptide antibody used in these flow cytometry and immunofluorescence experiments does not distinguish fully processed insulin from its precursor peptides. When assessing insulin secretion from these cells with a human processed insulin specific ELISA, polyhormonal cells do not secrete significant quantities of insulin as compared to human islets. These results suggest that polyhormonal cells express pro/insulin but do not secrete fully processed insulin. Given these results, it was hypothesized that polyhormonal cells do not appropriately process proinsulin into mature insulin.

To evaluate the ability of these polyhormonal cells to process proinsulin the expression of the prohormone converting enzymes PC1 and PC2 that are responsible for processing insulin and glucagon respectively was looked at. At a transcriptional level, polyhormonal cells express PC2 to a much higher degree than they express PCl. This suggests a defect in insulin processing in these cells and supports the idea that polyhormonal cells do not process proinsulin into mature insulin. These results suggest that the polyhormonal cells are not only similar to alpha cells in their transcriptional signature, but that they possess the hormone processing activity of an alpha cell.

Polyhormonal Cells Resolve to Alpha Cells after Transplantation

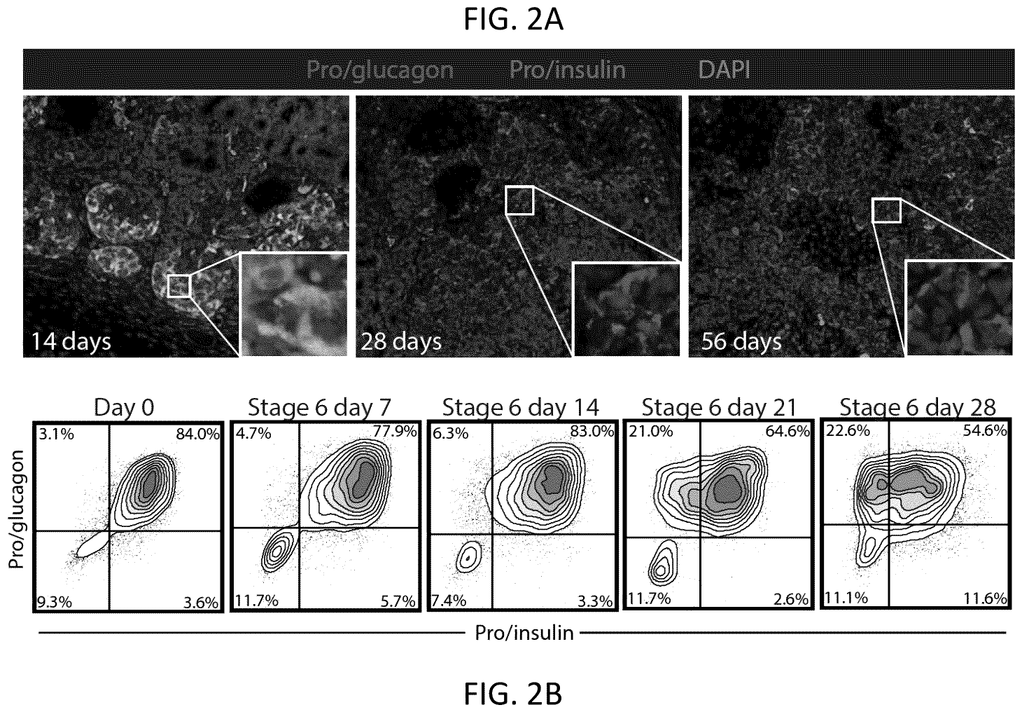

Previous studies have suggested that polyhormonal cells that appear early in development resolve into alpha cells at later developmental time points⁽²²⁻²⁴⁾. We wanted to test the ability of our polyhormonal cells to mature into alpha cells in vitro and in vivo. After transplantation into the kidney of immunocompromised mice, the polyhormonal cells, reduce the expression of pro/insulin over the course of 4 weeks. Polyhormonal cells were transplanted under the kidney capsule of SCID-beige mice. Grafts were retrieved 14, 28 or 56 days after transplantation and assessed for hormone expression using antibodies that react with pro/glucagon and pro/insulin. 14 days after transplantation, polyhormonal cells continue to express pro/glucagon and pro/insulin (FIG. 2A, left). When grafts were evaluated at 28 days, few c-peptide expressing cells were observed, while a number of pro/glucagon expressing cells persisted (FIG. 2A, middle). This population of monohormonal proglucagon expressing cells persisted for up to 56 days post-transplant (FIG. 2A, right). Serum glucagon levels in these animals were elevated compared to non-transplanted controls. These results suggest that pro/insulin expression can be eliminated from polyhormonal cells with extended time in vivo.

### Some Polyhormonal Cells can Convert to SC-Alpha Cells

Given the ability of polyhormonal cells to reduce pro/insulin expression after transplantation, we sought to test the ability of our polyhormonal cells to mature into monohormonal expressing cells in vitro. After completion of stage 5 of our protocol, we continued to culture our polyhormonal cell for an additional 4 weeks in media without growth factors. During this extended culture period, we sampled the cell at regular 7-day intervals and assessed the expression of pro/glucagon and pro/insulin by flow cytometry. As shown in FIG. 2B, the vast majority of these cells (>80%) expressed both pro/insulin and pro/glucagon at the beginning of this extended culture period. After 14 days of extended culture, little change had occurred in the expression levels of pro/insulin and pro/glucagon with >80% of the cells persisting in a polyhormonal state. After 21 days of extended culture, a population of pro/glucagon+, pro/insulin− cells begins to appear with ˜20% of the cells expressing pro/glucagon in a monohormonal fashion. This population of monohormonal alpha cells persists after 28 days of extended culture. These results indicate that upon extended culture in vitro, a fraction of polyhormonal cells are able to reduce their pro/insulin expression and become SC-alpha cells.

### PKC Activation Promotes Resolution of Polyhormonality and Supports Alpha Cell Identity

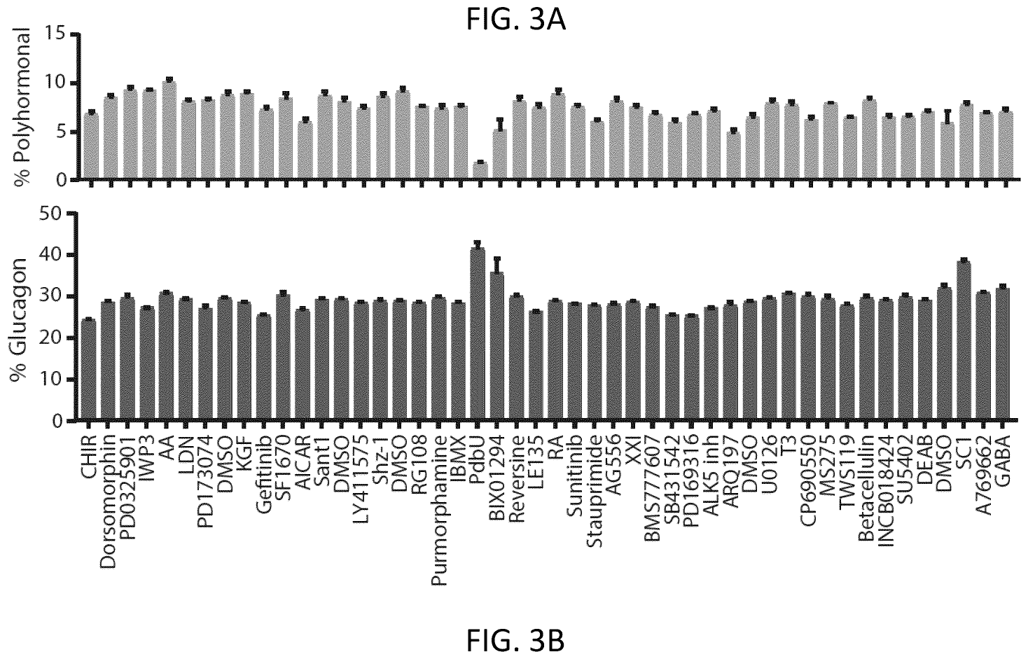

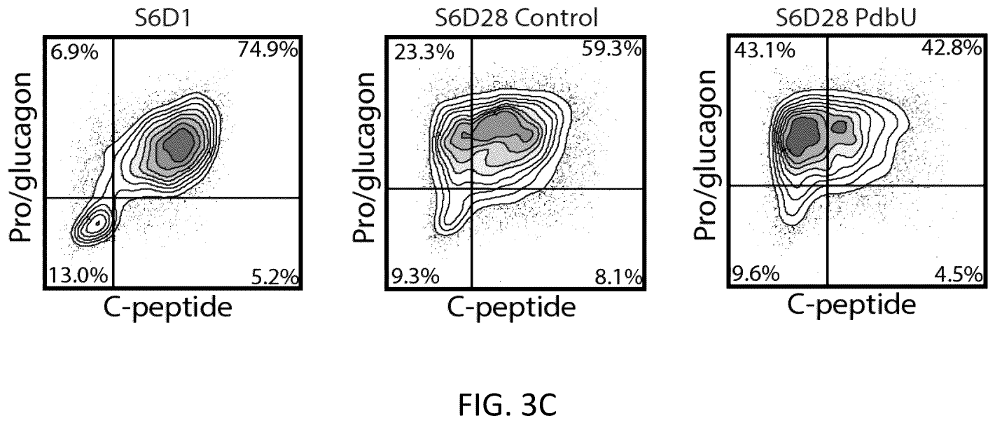

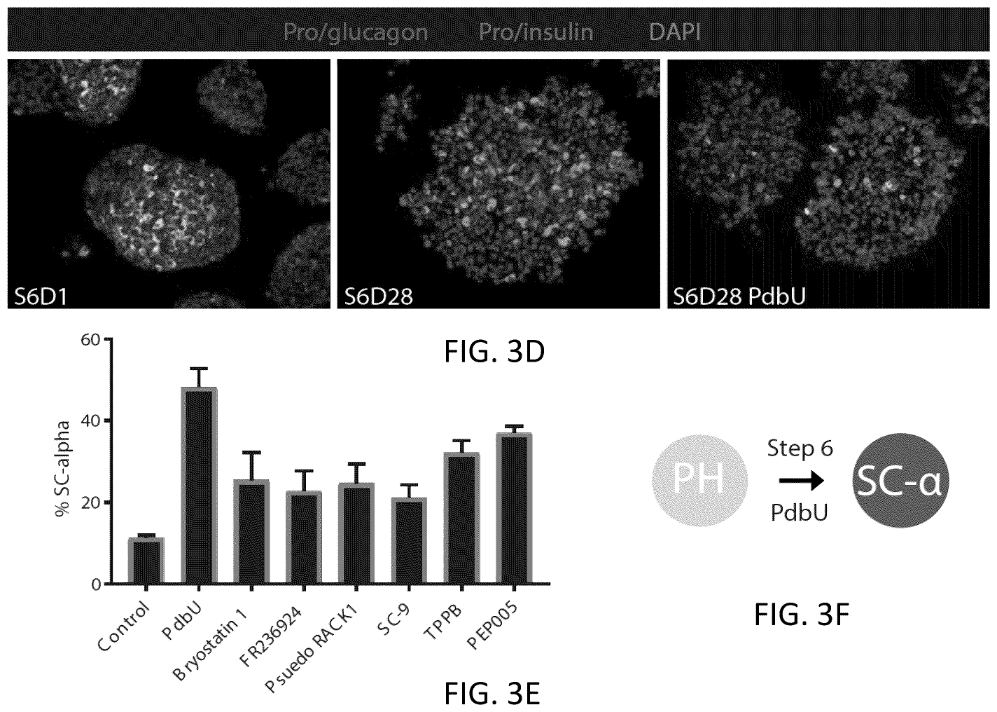

Having demonstrated the ability of these cells to reduce insulin expression and mature into SC-alpha cells, we sought to identify relevant signals that could promote a stronger conversion of polyhormonal cells into SC-alpha cells. Toward this goal, we initiated a small-molecule phenotypic screen to identify compounds that either promote alpha cell identity or conversely reduce proinsulin expression in vitro. Briefly, HUES8 cells were differentiated using our polyhormonal optimized protocol. Cells were dispersed and arrayed into a 384-well plate and compounds were introduced. HUES8-derived polyhormonal cells were co-incubated with a compound library for 96 hours, after which the cells were fixed and stained for pro/glucagon and pro/insulin. High-content imaging was used to quantify the percentage of cells expressing each hormone individually and the percentage of polyhormonal cells as marked by the co-expression of both hormones (FIGS. 3A-3B). In total 43 compounds were evaluated in quintuplicate. From these results, the effect of each compound on the polyhormonal population was evaluated. The PKC activator, PdbU was found to significantly decrease the percentage of pro/insulin expressing cells and conversely increase the percentage of pro/glucagon expressing cells. To confirm that the effects of this compound were not unique to the assay format, PdbU was evaluated in the final stage of the directed differentiation protocol. As shown in FIG. 3C, over the course of a 28 day treatment, PdbU induced a significant population of polyhormonal cells to reduce insulin expression (46±4% vs. 23±3%). Taken together, these results suggest a role for PKC activation in the repression of insulin expression. The resulting cell population contains significantly more alpha cells that do not co-express pro/insulin (FIG. 1D).

To test the specificity of PKC activation on the results shown above, we compiled a small library of known PKC activators and evaluated their ability to similarly decrease insulin expression in polyhormonal cells. This structurally diverse set of compounds has been reported to harbor PKC activating abilities. We sought to determine if these compounds would similarly be capable of promoting alpha cell identity and maturation in a polyhormonal population. To that end, we treated polyhormonal cells for 28 days with either vehicle or each PKC activator and assessed the percentage of monohormonal expressing alpha cells at the end of the 28-day treatment. While the control condition resulted in only 12% SC-alpha cells, treatment with each PKC activator resulted in a significant increase in the percentage of SC-alpha cells. These results demonstrate that activation of PKC resolves polyhormonality in stem cell derived cells and promotes alpha cell identity.

SC-Alpha Cells have Functional Similarities to Human Alpha Cells

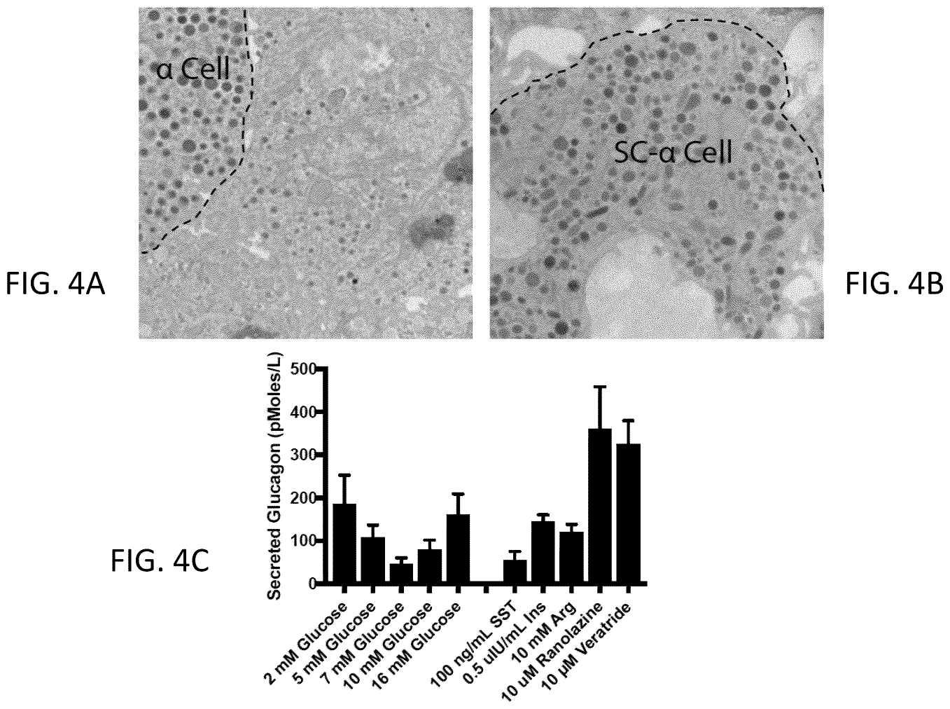

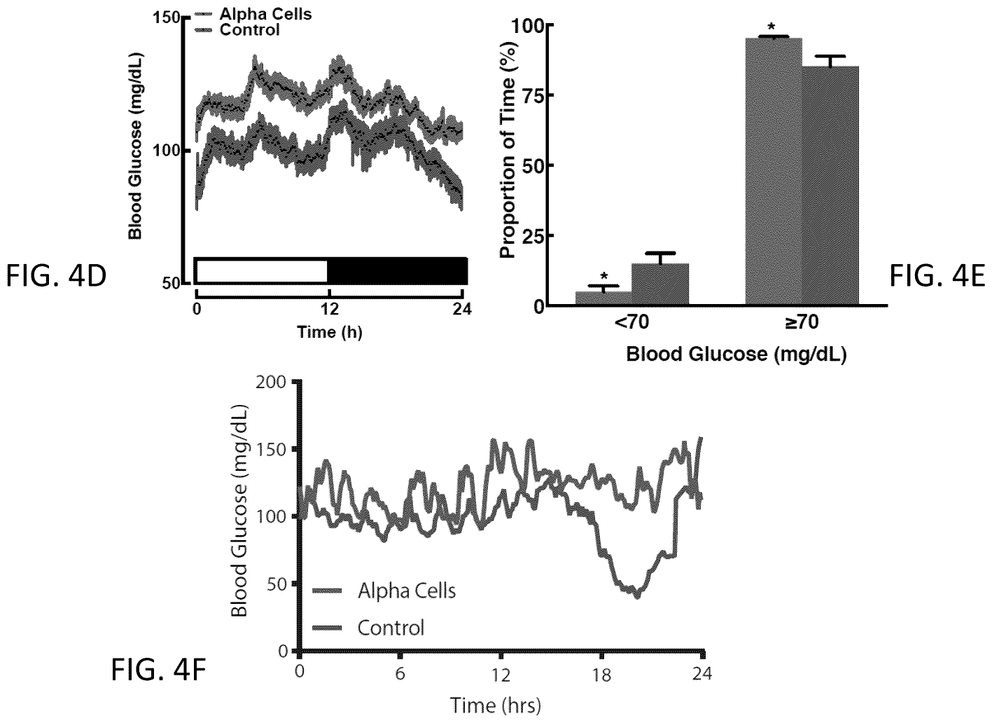

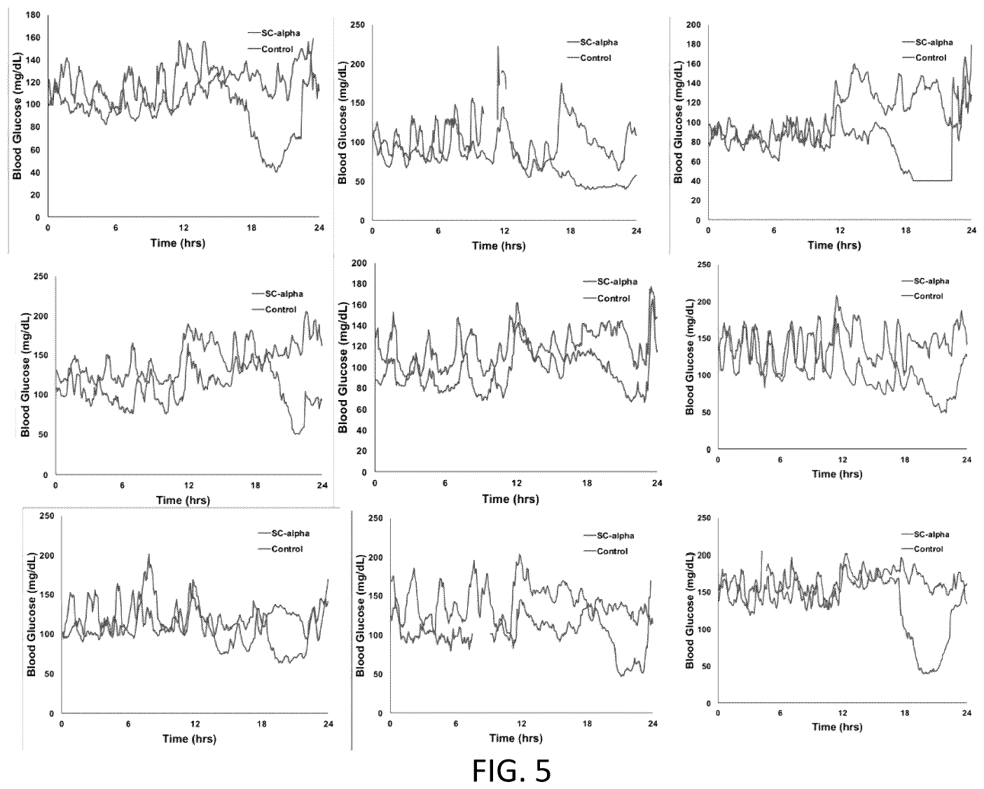

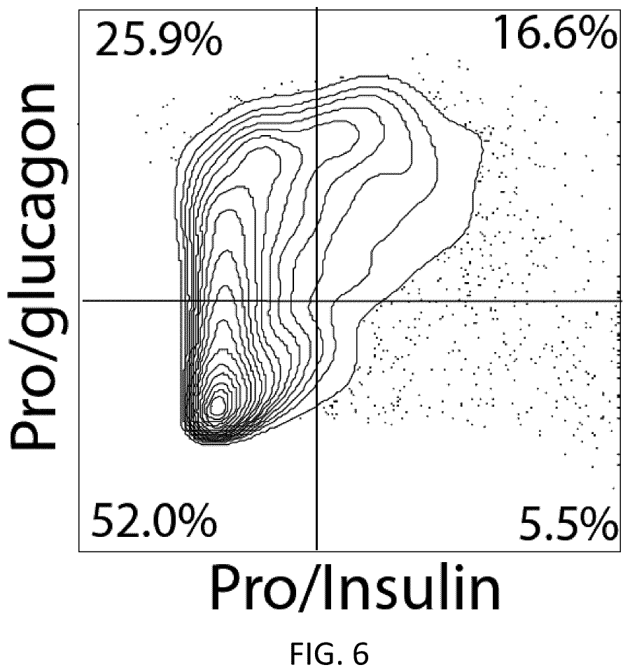

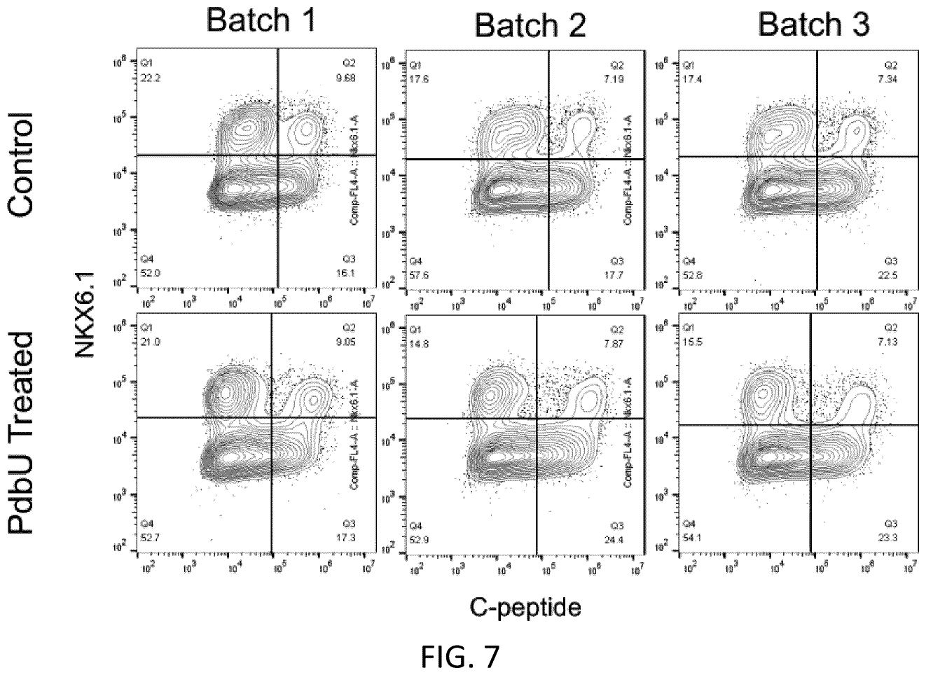

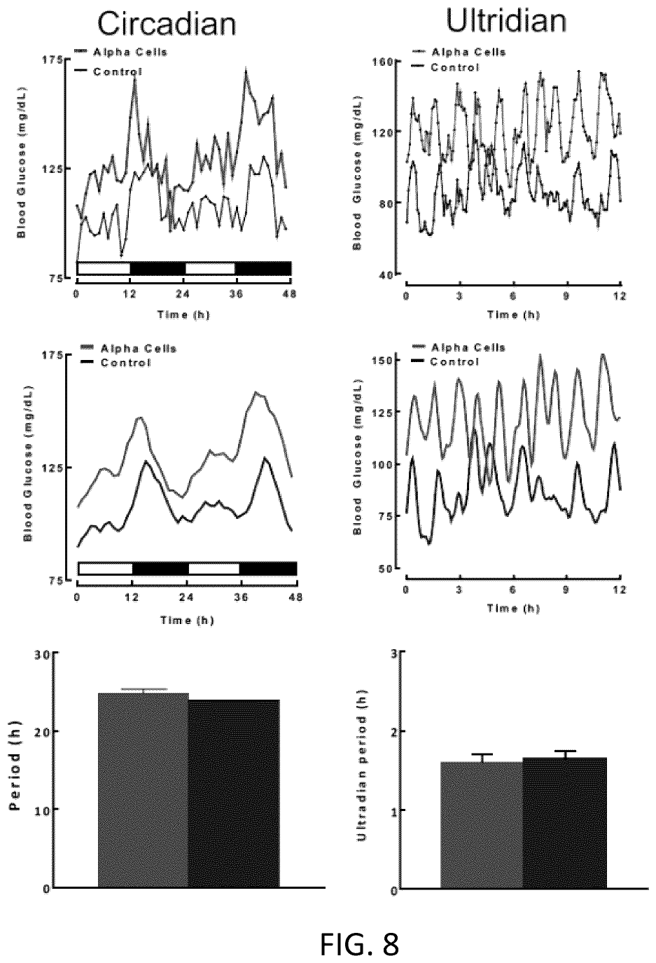

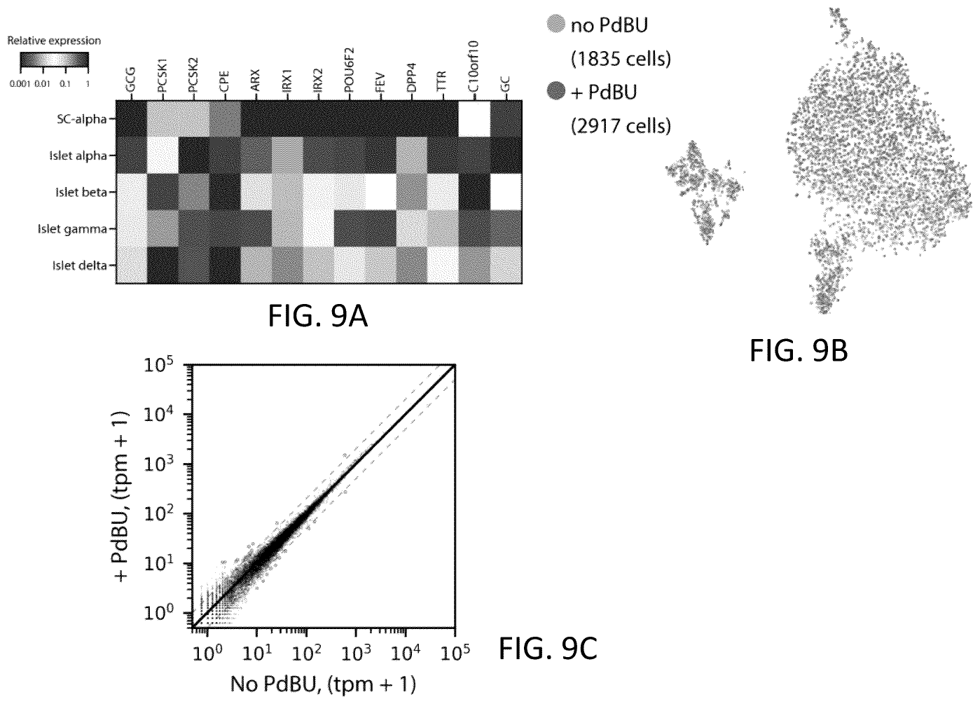

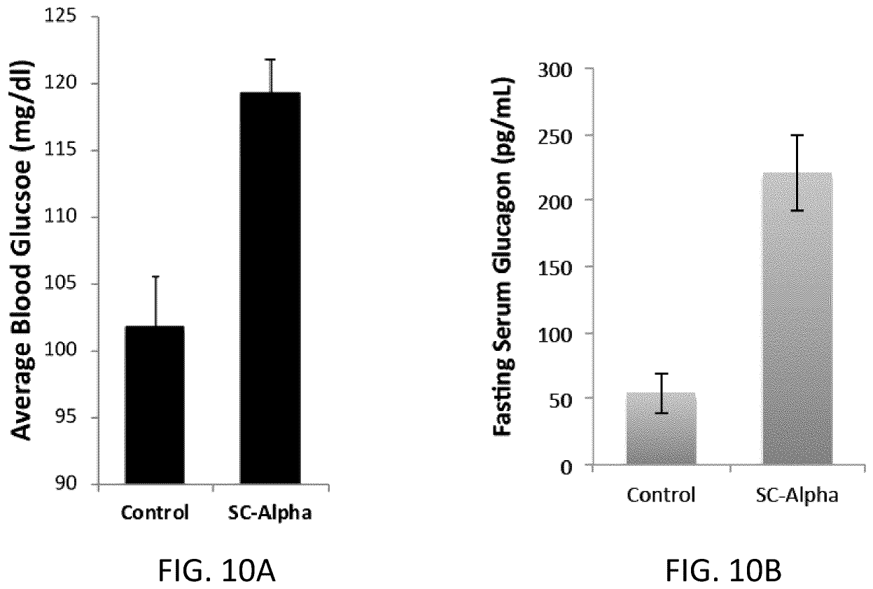

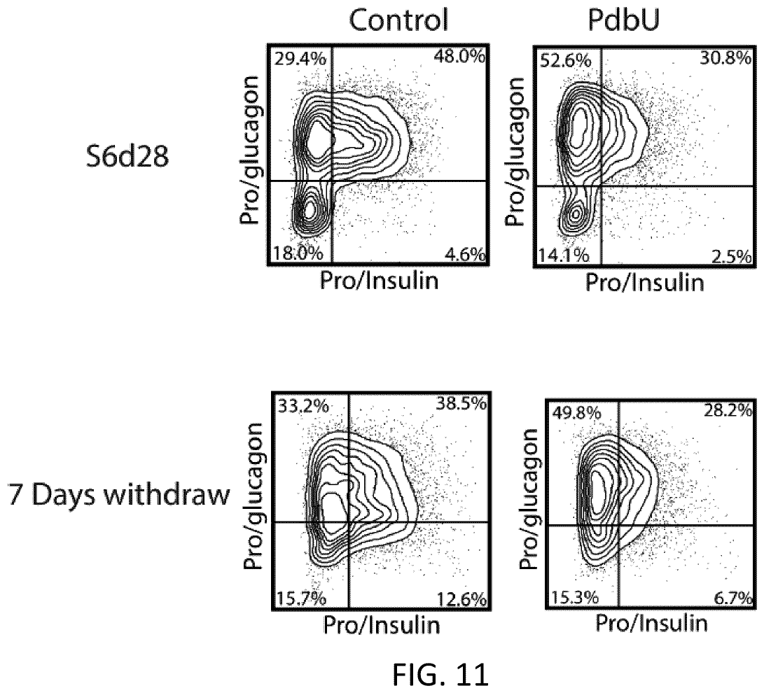

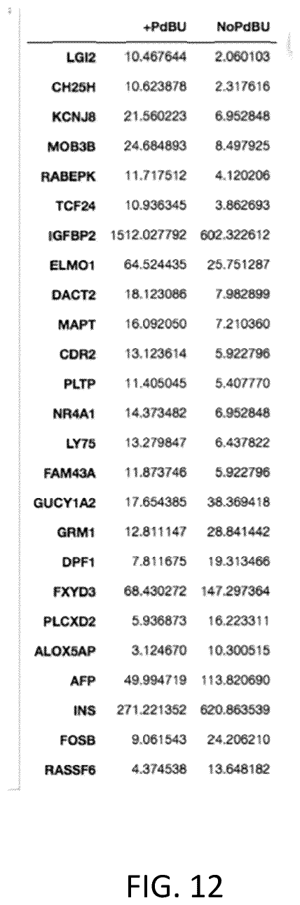

With a protocol in hand for the generation of SC-alpha cells we sought to characterize the resulting cell population. Using single cell RNAseq we profiled the transcriptome of SC-alpha cells (FIG. 4A). tSNE clustering reveals 3 populations in our culture. The predominant population contains transcripts for glucagon and insulin, however, the expression level of insulin is 3 orders of magnitude lower than the expression level of glucagon (mean tpm of 649 vs. 214,320). Further this population expresses additional transcription factors that are known markers of cadaveric alpha cells (ARX, IRX1, IRX2). Although there is a small population of cells that express the beta cell marker Nkx6.1, the SC-alpha population does not express Nkx6.1. Further the evaluation of the prohormone convertase enzymes supports the proper expression of PC2 for processing of glucagon while the expression of PC1 is significantly lower.

Electron Microscopy reveals that the ultrastructure of SC-alpha cells resembles that of human primary alpha cells. In human islets, glucagon granules in alpha cells are dark and diffuse with round cores. The average size of these granules is 240 μm which is larger than the condensed granules of the beta cell (FIG. 4B). In SC-alpha cells, the secretory granules have a similar morphology to alpha cells with an average size of 220 nm. These results suggest that SC-alpha cells can transcribe and processes glucagon into secretory granules.

To evaluate the ability of SC-alpha cells to respond to physiological conditions, we screened a number of glucagon secretion modulators and evaluated the effect on glucagon secretion. In the human islet, alpha cells secrete glucagon in response to low glucose conditions. Alpha cells have also been shown to respond to amino acids with increased glucagon secretion in response to mixed amino acids, even in high glucose conditions, which is a physiological response normally seen in response to intravenous administration of glucose and arginine in humans. Additionally, alpha cells respond to paracrine inhibitory signaling from delta cells through the hormone somatostatin resulting in suppressed glucagon secretion.

SC-alpha cells exhibit a similar response to these stimuli. As shown in FIG. 4c, glucagon secretion is suppressed in the presence of exogenous somatostatin. Further, SC-alpha cells respond to the administration of amino acids as shown in FIG. 4c. A number of pharmacological compounds have been developed to modulate glucagon secretion. We evaluated the effect of these compounds on SC-alpha cells to determine how well SC-alpha cells recapitulate the functional response of alpha cells (FIG. 4C). These results demonstrate that SC-alpha cells resemble human primary alpha cells in their transcriptional profile, glucagon granule morphology, and physiological response to glucagon secretion modulators.

Differentiation of hPSCs to beta cells results in a side population of polyhormonal cells. To this end we evaluated the effect of PKC activation on the differentiation of SC-beta cells. Treatment of stage 6 SC-beta cells with the PKC activator PdbU did not have a significant effect on the number of beta cells in the population. However, when treated with PdbU, the polyhormonal side population in the differentiation was reduced, and the percentage of monohormonal alpha cells was increased. These results suggest that in varied contexts, PKC activation is able to promote the conversion of polyhormonal cells to SC-alpha cells.

### SC-Alpha Cells Protect Against Hypoglycemia in Transplantation Models

Having developed a protocol for the generation of monohormonal expressing SC-alpha cells, we were interested in evaluating the potential utility of these cells to modulate physiology in vivo. To this end we transplanted SC-alpha cells under the kidney capsule of mice and evaluated the effect of these cells on mouse physiology. Using continuous glucose monitoring, we evaluated the glucose concentrations in animals at 5-minute intervals starting 4 weeks after transplantation and continuing for 4 weeks. During this time, mice were maintained under normal housing conditions with 24 hour light/dark cycles and feeding ad libitum. At the end of the 4-week observation period, glucose readings for all mice in a cohort were averaged and represented as an average blood glucose value per 5-minute interval throughout a standard 24-hour period with the associated variation (FIG. 4d). As has previously been reported, control mice exhibited a characteristic fluctuation pattern in blood glucose values over a standard 24-hour period with elevated blood glucose levels during active/feeding periods (dark) and lower blood glucose levels during resting periods (light). In transplanted mice we observed a similar pattern of glucose fluctuations, although the average glucose concentrations were elevated compared to control mice. This data shows that the transplantation of SC-alpha cells does not perturb the normal circadian regulation of blood glucose levels in these animals but that the presence of additional glucagon secreting cells raises the basal blood glucose levels in these animals. Further evaluation of circadian and ultridian patterns in blood glucose levels confirms that there is no significant shift in periodicity of these previously reported glucose rhythms. The effect of SC-alpha cells on blood glucose is further confirmed by the elevation of fasting blood glucose in these animals and the increase in circulating glucagon levels in animals transplanted with SC-alpha cells.

The sum result of SC-alpha cell transplantation is an elevation of blood glucose levels and a reduction in the proportion of time that animals spend with blood glucose levels in the hypoglycemic range (<70 mg/dl). As shown in FIG. 4E, mice that have received SC-alpha cell transplants spend on average 5% of the time in hypoglycemia as compared to 15% for control animals. These results suggest that transplantation of SC-alpha cells has the ability to protect animals from hypoglycemia. To further explore the ability of SC-alpha cells to protect against hypoglycemia in more extreme situations, we challenged mice with an exogenous insulin injection. As shown in FIG. 4F, control animals injected with exogenous insulin demonstrated a significant drop in blood glucose concentrations and became hypoglycemic. In contrast, animals that received a transplantation of SC-alpha cells were protected from this insulin induced hypoglycemia. These results highlight the potential of SC-alpha cells to regulate blood glucose levels in vivo and their potential as a therapeutic strategy to prevent hypoglycemia in type 1 diabetic patients.

### DISCUSSION

The promise of regenerative medicine lies in the ability to produce tissues in the laboratory to replace damaged or diseased tissues. Diabetes has long been a leading candidate for this approach because a single cell type, the beta cell, plays such a critical role in the disease. This has led to a nearly single-minded focus on the generation of pancreatic beta cells. Although much progress has been made on the generation of beta cells, we are now beginning to appreciate the potential need for other cell types to adequately treat diabetes. In this study, we have moved beyond the beta cell to focus our attention on the generation of pancreatic alpha cells that could be used to deepen our understanding of the normal functioning of the islet and perhaps develop cell-based therapies to prevent hypoglycemia. Recent studies have highlighted the importance of multiple islet cell types to the regulation of insulin secretion. Paracrine signaling from alpha cells is critical in establishing a set point for insulin secretion, and glucagon being critical for regeneration of beta cells in vivo. Taken together, these studies suggest that the generation of human islets that incorporate beta cells, alpha cells and potentially additional endocrine cell types will be important for cell replacement therapies. The generation of SC-alpha cells is an important step toward that goal and enables future studies looking at co-culture of alpha and beta cells to better understand the interplay between these cell types in islet function and disease.

An ideal cell replacement therapy rests on an adequate understanding of alpha cell dysfunction and a product that not only restores beta cell function, but also restores appropriate alpha cell function. Recent studies have suggested that alpha cell dysfunction plays a role in the chronic hyperglycemia seen in patients with T1D due to hyperglucagonemia⁽⁵⁾. Additionally, a defective alpha cell response to hypoglycemia is part of the impaired counter-regulatory response to hypoglycemia observed in T1D⁽⁸⁾. After cessation of insulin secretion, glucagon is the first line of defense against hypoglycemia, prior to activation of sympathetic responses. In T1D, insulin concentrations are dependent on insulin dosing while the sympathetic counter-regulatory response is impaired by prior hypoglycemia. Accordingly, interventions to mitigate the risk of hypoglycemia are urgently needed in T1D.

Key to providing a cell replacement therapy aimed at restoring alpha cell function, is an understanding of the mechanisms that result in alpha cell dysfunction—a knowledge gap that applies to both type 1 and type 2 diabetes. It has been proposed that alpha cells do not function properly in T1D because they do not have appropriate paracrine signaling from beta cells and other islet cells⁽²⁵⁾. If this is the cause of alpha cell dysfunction, we will need to develop a cell replacement therapy that at minimum contains both alpha and beta cells. A cell replacement strategy that contains only alpha cells or only beta cells will likely never effectively restore glucose control. We are motivated to study the role of alpha cells on the in vitro function of hES-derived beta cells because we believe that it will lead to fundamental new insights into our understanding of the function of the islet. Glucagon has been shown to play a stimulatory role in insulin secretion⁽²⁶⁾, but the precise role of this stimulatory effect has never been studied in an in vitro context. Even in the absence of exogenous beta cell transplantation, the transplantation of SC-alpha cells may provide a clinical benefit in protecting against hypoglycemia in T1D. We demonstrate that transplantation of SC-alpha cells could protect mice from hypoglycemia.

Despite mounting evidence that defects in beta cell function do not fully account for the dysregulation observed in diabetes^{(5, 8)}, current therapies for diabetes (both type 1 and type 2) have focused almost exclusively on the insulin producing beta cell. For example, insulin replacement therapy used in patients with type 1 and type 2 diabetes seeks to replace insulin that would normally be produced by beta cells. In type 2 diabetes, treatment with sulfonylureas such as glimepiride induce higher insulin secretion rates from pancreatic beta cells. Few therapies are currently on the market that target other cell types in the pancreas. GLP-1 receptor agonists such as exenatide have been shown to reduce glucagon secretion from the pancreas, but it is unclear if the mechanism for this effect includes direct effects on alpha cells since the expression of GLP-1 receptors on alpha cell is low and is more likely to be related to secondary effects like altered insulin and somatostatin secretion. Reduced glucagon levels have also been observed following treatment of type 2 diabetic patients with DPP-4 inhibitors, and since DPP-4 inhibition result in increased circulating endogenous GLP-1, a similar mechanism as for GLP-1 receptor agonists has been suggested. There have been multiple attempts to produce glucagon receptor antagonists, since the high glucagon levels observed in type 2 diabetics may contribute to the hyperglycemia and suggested to be one part of the pathophysiology, however this concept has failed in the clinic due to undesired side-effects like elevated plasma lipids and accumulation of liver fat. On the other hand, glucagon secretagogues have been reported such as veratridine and the TRG5 agonist Abt-777, that potentially could be used in treatment of hypoglycemia in type 1 diabetic patients.

In this study we describe a protocol for generating polyhormonal cells that express both pro/insulin and pro/glucagon. These polyhormonal cells have been described in a number of different situations including during development, in pancreatitis, under stress conditions, and as an unexpected side population in directed differentiation protocols. Confusion regarding the nature of these cells has existed with some reports describing these cells as immature beta cells, others describing these cells as intermediates for alpha cells and yet others describing these cells as a hybrid cell type with features of both alpha and beta cells. In our differentiations, polyhormonal cells do not represent a hybrid cell type but rather have a transcriptional signature reminiscent of an alpha cell. Polyhormonal cells have been observed early in mouse development. Despite these reports of polyhormonal cells, have not been observed in the healthy adult pancreas. Our studies have demonstrated the potential of human polyhormonal cells to mature into alpha cells. Additionally, we have identified an important pathway involved in this maturation process, the activation of PKC.

In this series of experiments, activation of PKC resulted in the maturation of polyhormonal cells into glucagon expressing SC-alpha cells. PKC activation has been reported to enhance glucagon secretion from rat and human islets through calcium dependent exocytosis. Further a role for PKC in the transcription and expression of glucagon has been reported. Our studies further establish the importance of PKC in alpha cells and establishes a new role for PKC activation in alpha cell identity and maturation. While further studies will be needed to identify the precise mechanistic action of PKC activation alpha cell maturation, these studies firmly establish new avenues for investigation. These studies, as well as studies aimed at combining SC-alpha and SC-beta cells will be the subject of future reports from our group.

### Materials and Methods

### Cell Culture

Human pluripotent stem cells (HUES8 and 1016 cell lines) were maintained as previously described⁽¹⁰⁾. Briefly, cells were adapted to 3D culture in spinner flasks and maintained using mTeSR media. Suspension culture was maintained at 70 rpm in a humidified incubator at 37° C. and 5% CO₂. Cells were passaged every 72 hours by dispersing to single cells using Accutase and seeded into fresh mTeSR with 10 μM Y27632. Media was changed at 48 hours to mTeSR without Y27632.

### Directed Differentiation

Differentiations were initiated 72 hours after seeding into mTeSR. Clusters were allowed to settle to the bottom of the spinner flask and the media was removed by aspiration. Protocol specific media was introduced to the spinner flask with appropriate growth factors and the flask was returned to the incubator with stirring. Cells were directed sequentially to a DE, GTE, PP, EP and PH population as described below. Media changes are as follows—Day 1: S1+100 ng/ml Activin A+3 uM CHIR99021. Day 2: S1+100 ng/ml Activin A. Day 4: S2+50 ng/ml KGF. Day 6: S3+2 uM RA. Day 7: S3+2 uM RA+200 nM LDN193189. Day 8: S3+200 nM LDN193189. Day 9, 11: S3 (no additional factors). Day 13, 15, 17, 19: S3+10 uM Alk5i. Day 20-48: S3+500 nM PdbU (feed on even days only).

### Flow Cytometry

Cells were collected for flow cytometry analysis at various time points throughout the differentiation process. Cells were dispersed with TrypLE Express at 37° C. for 15 minutes after which trypsin was quenched with serum containing media (S1, S2, or S3). Cells were fixed in 4% PFA for 20 minutes and subsequently stored in PBS at 4° C. until staining. Staining was performed by first permeabilizing the cells in block solution (PBS+0.1% Triton X-100+5% donkey serum) for 40 minutes. Cells were then incubated with primary antibodies in block solution for 1 hour at RT, washed twice with PBST and incubated with secondary antibody in block solution for 1 hour at RT. Cells were then washed in three times and resuspended in PBST at a concentration of 1×106 cells/ml. Stained cells were analyzed using the Accuri C6 or Attune flow cytometers. Antibody concentrations used were as follows. Pro/insulin was assessed using an antibody that recognizes the c-peptide epitope from the Iowa Hybridoma Bank and used at a concentration of 1:300. Pro/glucagon was assessed using an antibody that recognizes the GLP-2 (Santa Cruz) or glucagon (Manufacturer) epitopes and used at a concentration of 1:300. Secondary antibodies used were a donkey-anti-goat-Alexaflour 488 (1:500) and a donkey-anti-rat-Alexaflour 647 (1:500).

### Immunofluorescence

Cell clusters or kidney grafts were fixed in 4% PFA, washed and embedded in Histogel (clusters) and subsequently embedded in paraffin and sectioned. Sections were stained for pro/insulin and pro/glucagon using the antibodies described above and mounted using Flourmout-G with DAPI. Clusters were imaged using an EVOS FL Auto 2 imaging station.

### Single Cell RNA Sequencing

Single-cell mRNA sequencing as carried out using the inDrops platform^{(11, 12)}, as previously described using ‘v3’ beads (1-cell bio). Approximately 5000 cells were collected from both PdBu+ and PdBu− negative conditions. Two libraries were prepared from each sample using the inDrops protocol and sequenced on a NextSeq 500. Sequencing was processed using the inDrops pipeline (https://github.com/indrops/indrops/) to generate UMIFM read counts. Read counts were converted to tpm values by dividing the counts from each cell by the sum of all reads for that cell, excluding genes that contribute more than 2% of reads in any given cell. High-variance genes were identified as previously described⁽¹¹⁾ and their tpm values were use for principal component analysis (PCA). tSNE projection was carried out using the first 25 components (not rescaled to unit variance), using the Python wrapper of the Barnes-Hut C tSNE implementation (github.com/lvdmaaten/bhtsne). Clustering was performed on the using the principal components as input for diffusion map embedding and Louvain community detection, as implemented in Scanpy⁽¹³⁾.

### Hormone Secretion Assays

Human islets or differentiated polyhormonal or SC-alpha cells were washed twice in low-glucose (2.8 mM) Krebs Ringer (KRB) buffer (128 mM NaCl, 5 mM KCl, 2.7 mM CaCl₂, 1.2 mM MgSO₄, 1 mM Na₂HPO₄, 1.2 mM KH₂PO₄, 5 mM NaHCO₃, 10 mM HEPES, 0.1% BSA in milliQ). Cells were then loaded into 24 well transwell inserts and fasted in low glucose KRB for 1 hour at 37° C. Clusters were washed once in low glucose KRB and then incubated in low glucose KRB for 1 hour at 37° C. After incubation, the supernatant was collected and stored at −20° C. until analysis. Cells were then transferred to high glucose KRB (20 mM) for 1 hour at 37° C. and the supernatant was collected and stored. Cells were then transferred to low glucose KRB with 30 mM KCl to observe depolarization conditions. Cells were incubated in this buffer for 1 hour and the supernatant was collected. Cells were then dispersed and counted using a vicell automated cell counter. Collected supernatants were analyzed by ELISA for Insulin and/or glucagon and normalized to cell number.

### Chemical Screen

Polyhormonal cells were dispersed using TrypLE Express for 15 minutes and quenched using S3 media. The resulting single cell suspension was arrayed into a 384 well plate which had been coated with Matrigel with 50,000 cells in each well. Compounds were introduced each to 5 separate wells and incubated for 96 hours after which cells were fixed with 4% PFA and stained for pro/insulin and pro/glucagon as described above with the following modifications. Antibody incubations and washes were performed using a BioTek EL405 Plate washer and dispenser. Cells were imaged with the cellomics array scan high content imaging system. Nine fields of view were captured for each well and analyzed for cell number and fluorescent content. The percentage of cells expressing pro/insulin and pro/glucagon was calculated for each well.

### Electron Microscopy

To analyze the granule ultrastructure, human islets or differentiated SC-alpha cells were fixed for 2 hours at RT in fixative (2.5% Glutaraldehyde 1.25% Paraformaldehyde and 0.03% picric acid in 0.1 M sodium cacodylate buffer, pH 7.4) washed in 0.1M cacodylate buffer and postfixed with 1% Osmiumtetroxide (OsO4)/1.5% Potassiumferrocyanide(KFeCN₆) for 1 hour, washed 2× in water, 1× Maleate buffer (MB) 1× and incubated in 1% uranyl acetate in MB for 1 hr followed by 2 washes in water and subsequent dehydration in grades of alcohol (10 min each; 50%, 70%, 90%, 2×10 min 100%). The samples were then put in propyleneoxide for 1 hr and infiltrated overnight in a 1:1 mixture of propyleneoxide and TAAB Epon (Marivac Canada Inc. St. Laurent, Canada). The following day the samples were embedded in TAAB Epon and polymerized at 60° C. for 48 hrs.

### Cell Transplantation

Transplantation of cell clusters was performed as previously described⁽¹⁰⁾. Briefly, 5×106 cells were injected under the kidney capsule of SCID-beige mice. Mice were monitored for up to 8 weeks after transplantation. Kidney grafts were harvested at various time points and stained for pro/insulin and pro/glucagon as described above.

### Continuous Glucose Monitoring

Transplanted mice were monitored using a Dexcom G5 continuous glucose monitoring (CGM) system. Each mouse was anesthetized with isoflurane prior to application of the CGM sensor and transmitter. Sensors were inserted into the subcutaneous space on the back of the mice and attached using wound clips. Sensors were calibrated twice a day using a handheld glucometer (Accucheck). Sensors were replaced every 7 days throughout the study (4 weeks).

### Data Analysis and Calculations

Data processing of the CGM-measured glucose concentrations was conducted using MATLAB 2015 (MathWorks, Natick, MA). The ultradian period of the CGM-measured glucose rhythms was quantified using autocorrelation analysis as previously described for the characterization of pulsatile insulin secretion⁽¹⁴⁾. MATLAB 2015 was used to calculate correlation coefficients of the CGM-measured glucose concentrations. The dominant circadian period in glucose concentration was assessed using Lomb-Scargle periodogram analysis (ClockLab, Actimetrics, Evanston, IL, USA). Statistical analysis was performed using either ANOVA or Student's unpaired t test, where appropriate (GraphPad Prism v.6.0). Data was presented as mean±SEM and considered statistically significant at p<0.05.

### REFERENCES

1. M. Brissova, et al., Cell Rep 22, 2667-2676 (2018).
2. R. H. Unger, et al., J Clin Invest 122, 4-12 (2012).
3. A. M. Leckie, et al., Diabetes Care 28, 1333-1338 (2005).
4. B. M. Frier, Diabetes Res Clin Pract 65 Suppl 1, S47-52 (2004).
5. G. Jiang, et al., Am J Physiol Endocrinol Metab 284, E671-678 (2003).
6. E. Mutel, et al., Diabetes 60, 3121-3131 (2011).
7. R. A. Fanne, et al., J Neurosurg 114, 85-91 (2011).
8. A. Siafarikas, et al., Diabetes Care 35, 1757-1762 (2012).
9. A. Rezania, et al., Diabetes 60, 239-247 (2011).
10. F. W. Pagliuca, et al., Cell 159, 428-439 (2014).
11. A. M. Klein, et al., Cell 161, 1187-1201 (2015).
12. R. Zilionis, et al., Nat Protoc 12, 44-73 (2017).
13. F. A. Wolf, et al., Genome Biol 19, 15 (2018).
14. D. Lang, et al., New England Journal of Medicine 301, 1023-1027 (1979).
15. O. G. Kelly, et al., Nat Biotechnol 29, 750-756 (2011).
16. J. E. Bruin, et al., Stem Cell Res 12, 194-208 (2014).
17. H. A. Russ, et al., EMBO J 34, 1759-1772 (2015).
18. T. van der Meulen, et al., Rev Diabet Stud 11, 115-132 (2014).
19. A. Rezania, et al., Stem Cells 31, 2432-2442 (2013).
20. K. Osafune, et al., Nat Biotechnol 26, 313-315 (2008).
21. M. Baron, et al., Cell Syst 3, 346-360 e344 (2016).
22. M. J. Riedel, et al., Diabetologia 55, 372-381 (2012).
23. M. Riopel, et al., Islets 6, e982949 (2014).
24. T. Hashimoto, et al., Anat Embryol (Berl) 178, 489-497 (1988).
25. G. J. Taborsky, Jr., et al., Diabetes 47, 995-1005 (1998).
26. K. Moens, et al., Diabetes 47, 66-72 (1998).

What is claimed is:

1. A method of producing an SC-alpha cell from a polyhormonal cell, the method comprising contacting a population of cells comprising a polyhormonal cell with PdbU to induce the maturation of at least one polyhormonal cell in the population into an SC-alpha cell.

2. The method according to claim 1, wherein the SC-alpha cells express and secrete glucagon in response to glucose and glucagon secretagogues.

3. The method according to claim 1, wherein the SC-alpha cells exhibit an ultrastructure similar to cadaveric alpha cells.

4. A method of producing an SC-alpha cell from a stem cell in vitro, the method comprising:
a) contacting a population of cells comprising a gut tube cell with i) at least one bone morphogenic protein (BMP) signaling pathway inhibitor and ii) at least one retinoic acid (RA) signaling pathway activator, to induce the differentiation of at least some of the gut tube cells into pancreatic progenitor cells;
b) contacting a population of cells comprising a pancreatic progenitor cell with at least one BMP signaling pathway inhibitor, to induce the differentiation of at least some of the pancreatic progenitor cells into endocrine progenitor cells;
c) contacting a population of cells comprising a endocrine progenitor cell with at least one TGF-β signaling pathway inhibitor, to induce the differentiation of at least some of the endocrine progenitor cells into polyhormonal cells; and
d) contacting a population of cells comprising a polyhormonal cell with Pdbu to induce the maturation of at least one polyhormal cell into at least one SC-alpha cell.

5. The method of claim 4, wherein the BMP signaling pathway inhibitor comprises LDN193189,
wherein the RA signaling pathway activator comprises retinoic acid, and/or
wherein the TGF-β signaling pathway inhibitor comprises Alk5 inhibitor II

6. The method of claim 4, wherein the SC-alpha cell expresses one or more of the following genes: ARX, IRX1, IRX2, DPP4, GCG, PSCK2, Pou6F2, FEV, TTR, and GC.

7. The method of claim 4, wherein the expression of the genes is enriched relative to in vivo pancreatic populations.

8. The method of claim 4, wherein the SC-alpha cell secretes glucagon in response to glucose and/or glucagon secretagogues.

9. An SC-alpha cell produced by a method of claim 4, wherein the SC-alpha cell secretes glucagon in response to arginine at a lower level than a native alpha cell in response to arginine.

10. An SC-islet comprising one or more SC-alpha cells produced by a method of claim 4, wherein the SC-alpha cells secrete glucagon in response to arginine at a lower level than a native alpha cell in response to arginine; optionally further comprising one or more of SC-β cells and SC-δ cells.

11. A method of producing an SC-alpha cell from a stem cell in vitro, the method comprising:
a) contacting a population of cells comprising a gut tube cell with i) LDN193189 or analogs or derivatives thereof and ii) at least one retinoic acid (RA) signaling pathway activator, to induce the differentiation of at least some of the gut tube cells into pancreatic progenitor cells;
b) contacting a population of cells comprising the pancreatic progenitor cells with LDN193189 or analogs or derivatives thereof, to induce the differentiation of at least some of the pancreatic progenitor cells into endocrine progenitor cells;
c) contacting a population of cells comprising the endocrine progenitor cells with at least one TGF-β signaling pathway inhibitor, to induce the differentiation of at least some of the endocrine progenitor cells into polyhormonal cells; and
d) contacting a population of cells comprising the polyhormonal cells with PdbU to induce the maturation of at least one polyhormal cell into at least one SC-alpha cell.

12. The method of claim 11, wherein the RA signaling pathway activator comprises retinoic acid and/or the TGF-β signaling pathway inhibitor comprises Alk5 inhibitor II.

13. The method of claim 11, wherein the SC-alpha cell expresses one or more of the following genes: ARX, IRX1, IRX2, DPP4, GCG, PSCK2, Pou6F2, FEV, TTR, and GC.

14. The method of claim 11, wherein the SC-alpha cell co-expresses the genes ARX, IRX1, and IRX2.

15. The method of claim 11, wherein the expression of the genes is enriched relative to in vivo pancreatic populations.

16. The method of claim 11, wherein the SC-alpha cell secretes glucagon in response to glucose and/or glucagon secretagogues.

17. The method of claim 11, wherein (i) the SC-alpha cell does not express c-peptide; and/or (ii) the SC-alpha cell is monohormonal; and/or (iii) the SC-alpha cell comprises one or more glucagon granules; and/or (iv) the SC-alpha cell exhibits an ultrastructure similar to cadaveric alpha cells.

18. The method of claim 11, wherein the SC-alpha cell is human.

19. An SC-alpha cell produced by a method of claim 11, wherein the SC-alpha cell secretes glucagon in response to arginine at a lower level than a native alpha cell in response to arginine.

20. An SC-islet comprising one or more SC-alpha cells produced by a method of claim 11, wherein the SC-alpha cells secrete glucagon in response to arginine at a lower level than a native alpha cell in response to arginine; optionally further comprising one or more of SC-β cells and SC-δ cells.
